(19) United States
(12) Patent Application Publication
(10) Pub. No.: US 2003/0186867 A1
(43) Pub. Date: Oct. 2, 2003

(54) Use of crf receptor agonists for the treatment or prophylaxis of diseases, for example neurodegenerative diseases

(76) Inventors: Laura Facci, Harlow Essex (GB); Stephen Drake Skaper, Harlow (GB); Paul Johannes Leonardus Maria Strijbos, Harlow Essex (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/240,429

(22) PCT Filed: Mar. 27, 2001
(86) PCT No.: PCT/GB01/01351

(30) Foreign Application Priority Data
Mar. 31, 2000 (GB) 0007856.8
Dec. 19, 2000 (GB) 0031067.2

(51) Int. Cl. A61K 38/17

(52) U.S. Cl. 514/12

(57) ABSTRACT

CRF receptor agonists, especially CRF receptor-1 agonists such as CRF, urocortin, sauvagine or urotensin 1, can be used for the prevention or inhibition of neuronal cell death in a mammal suffering from or susceptible to chronic neurodegenerative disease (e.g. Alzheimer's disease, Parkinson's disease or Huntington's disease), traumatic (mechanical) neuronal injury, epilepsy-associated neuronal loss, paralysis, or spinal chord injury. CRF receptor-1 agonists can also be administered to aid the prevention or inhibition of neuronal cell death in a mammal suffering from or susceptible to cerebral ischaemia (stroke). Also, where neuronal cell death is potentiated by inhibition or suppression of the PI 3-kinase signalling pathway, a treatment comprises administering to the mammal an effective amount of a CRF receptor agonist.

The present invention relates to the uses of CRF receptor agonists for the treatment or prophylaxis of certain diseases, to methods of treatment of those diseases using CRF receptor agonists, and to CRF receptor agonists for use in the treatment of these diseases.

Corticotropin-releasing factor (CRF) is a 41 amino-acid peptide distributed broadly within the central nervous system (CNS) including the cerebellum, where its receptors have also been described. CRF is secreted by the hypothalamus in response to stress and stimulates the corticotrope cells of the anterior pituitary to release the hormone corticotropin (or adrenocorticotropic hormone, ACTH). ACTH binds to receptors in the adrenal cortex and activates the release of glucocorticoid hormones. CRF from ovine hypothalamus was first isolated and disclosed in U.S. Pat. No. 4,415,558 (Salk Institute) and in W. Vale et al., Science, 213, 1394-1397, 1981, and CRF from rat hypothalamus was disclosed in U.S. Pat. No. 4,489,163 (Salk Institute); potential uses of CRF in elevating levels of ACTH or β-endorphin, lowering blood pressure, elevating mood, and improving memory and learning are also suggested. The cognition-enhancing effects of CRF in rats were confirmed in Behan et al., Nature, 378, 284-287, 1995, but the use of a CRF receptor agonist for the treatment of the cognitive deficits seen in Alzheimer's disease was discouraged owing to its perceived associated side effects (the doses of CRF which produced increases in learning and memory also produced anxiety in rats). CRF stimulates cAMP production (Battaglia, G., et al, Synapse (1987) 1:572-581).

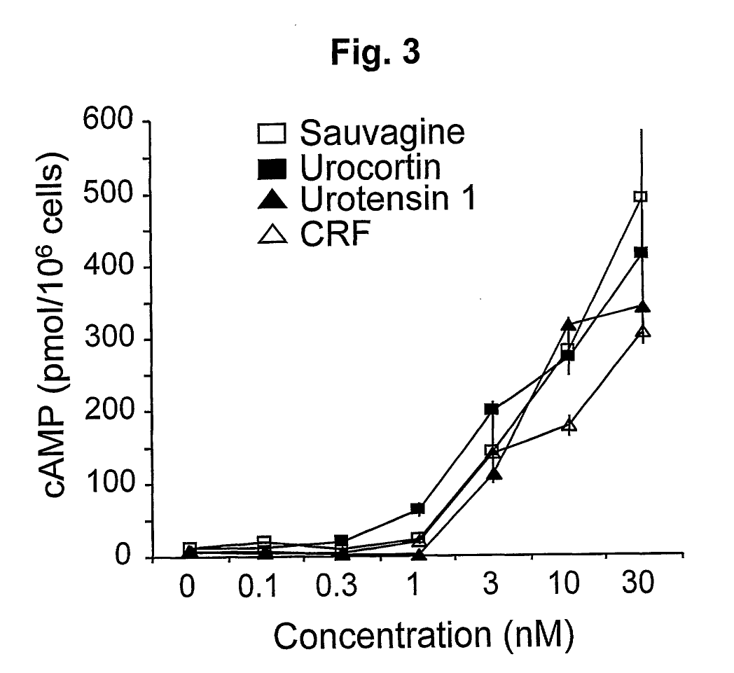

CRF has been shown to increase the excitability and spontaneous discharge frequency of hippocampal neurons (J. Aldenhoff et al., Science, 221, 875-877, 1983) and has been suggested but not proven to contribute to neurological injury during isehaemic or hypoxic insults (NM. Lyons et al., Brain Res., 545, 339-342, 1991; P. J. L. M. Strijbos et al., Brain Res., 656, 405-408, 1994). In contrast, in other experiments (M. W. Fox et al., Stroke, 24, 1072-1076, 1993), when rat hippocampus was subjected to a 10-minute hypoxic episode in the presence of glucose and either CRF or α-helical CRF 9-41 (α-CRF , a CRF antagonist), there was a dose-dependent recovery of synaptic function, as measured by extracellular recording of population spikes, in comparison to hypoxic controls. The Fox results were interpreted by the authors as suggesting that CRF may act as an endogenous neuroprotective hormone during hypoxia, though the mechanism of action was stated as being unknown and unclear as both CRF and the CRF antagonist gave similar results. In fact, these Fox results are characterised by confusion as to the mechanism by which CRF and α-CRF were acting. The authors said that further investigation of the effects of CRF and α-CRF was necessary to better define their mechanisms of action and determine their potential clinical roles in the treatment of cerebral ischaemia. An important caveat to the Fox paper is that it only measured the recovery from the electrical “silencing” of neurones, not protection from neuronal cell death; and the skilled person would know that no appreciable cell death would occur after 10 mins hypoxia but only after 60 mins of combined hypoxia and hypoglycaemia (glucose deprivation) (e.g. see A. K. Pringle et al., Brain Res., 755, 36-46, 1997, see especially p36 and FIG. 3B).

CRF receptors characterised so far are encoded by two distinct genes and differ in their anatomical distribution and affinities for CRF and other peptide CRF analogues. The Type 1 CRF receptor (CRF receptor-1 or CRF-R1) was isolated from rat/human pituitary/brain (R. Chen et al., Proc. Natl. Acad. Sci USA, 90, 8967-8971, 1993 (human brain); N. Vita et al., FEBS Lett., 335, 1-5, 1993 (human brain and mouse pituitary); M. H. Perrin et al., Endocrinology, 133, 3058-3061, 1993; C. Chang et al., Neuron, 11, 1187-1195, 1993) and appears to be concentrated in neocortical, cerebellar and sensory relay structures in rat brain (WO 95/34651, Neurocrine Biosciences, Inc.). CRF-R1 deficient mice have been disclosed (WO 99/50657).

A Type 2 CRF receptor (CRF receptor-2, CRF-R2) has been cloned from rat brain (WO 95/34651; and T. W. Lovenberg et al., Proc. Natl. Acad. Sci. USA., 92, 836-840, 1995) and mouse heart. One CRF-R2 subtype (splice variant) with 411 amino acids (CRF-R2α) and present in rats and humans is expressed in limited areas of the brain including the lateral septal, ventromedial hypothalamic, paraventricular and medial amygdaloid nuclei, and displays a much more restricted distribution than CRF-R1. Another 431-amino acid CRF-R2 splice variant (CR-R2β) is found in rodents in the brain adjacent arterioles, but mainly in the heart and skeletal muscle, and, although originally thought not to occur in humans, appears to be expressed in very low levels in e.g. human heart and skeletal tissues. A third CRF-R2 splice variant found in human brain is CRF-2γ (CRF-2c), exibiting pharmacology similar to CRF-R2γ. For references, see: N. Suman-Chauchan et al., Eur. J. Pharmacol., 379, 219-227, 1999; W. A. Kostich et al., Mol. Endocrinol., 12(8), 1077-1085, 1998 and Soc. Neurosci. Abstr, 22(2), 1545, 1996; O. Valdenaire et al., Biochim. Biophys. Acta, 1352(2), 129-132, 1997; RBI Handbook of Receptor Classification and Signal Transduction, ed. K. J. Watling, 3rd edition and any later edition; D. E Grigoriadis, T. W. Lovenberg, D. T. Chalmers et al., in Neuropeptides: Basic and Clinical Advances, Proceedings of the 5th Annual Summer Neuropeptide Conference, vol. 780, pp. 60-80, New York Academy of Sciences (1996); WO 95/34651 (Neurocrine Biosciences, Inc.); T. W. Lovenberg et al., Proc. Natl. Acad. Sci. USA., 92, 836-840, 1995; T. W. Lovenberg et al., Endocrinology, 136, 3351-3355, 1995; T. W. Lovenberg et al., Endocrinology, 136,4139-4142, 1995; C. W. Liaw, T. W. Lovenberg et al., Endocrinology, 137, 1996, 72-77; M. Perrin et al., Proc. Natl. Acad. Sci. USA, 92, 2969-2973, 1995; and E. Potter, Proc. Natl. Acad. Sci. USA, 91, 8777-8781, 1994; and references cited in any of these references.

Various CRF analogues are known which bind to and agonise (activate) CRF receptors. Sauvagine is a 40-amino-acid peptide related to CRF isolated from frog which stimulates ACTH and endorphin release and suppresses the suckling-induced rise of prolactin in lactating rats (P. C. Montecucchi and A. Henschen, Int. J. Peptide Protein Res., 18, 113, 1981; V. Espamer et al., Regulatory Peptides, vol. 2, (1981), pp 1-13; V. Erspamer and P. Melchiorri, Trends Pharmacol. Sci., 2, 391, 1980; P. Falaschi et al., Horm. Res., 13, 329, 1980; P. Falaschi et al., Endocrinology, 111, 693-695, 1982). Urotensin I is another peptide related to CRF which was purified and characterised from suckerfish by Lederis et al., Science, 218, 162-164, 1982. Both sauvagine and urotensin I bind to CRF-R1, CRF-R2α and CRF-R2β, and activate these receptors as measured by production of cAMP (cyclic adenosine monophosphate) (J. Vaughan et al., Nature, 378, 287-292, 1995; C. J. Donaldson et al., Endocrinology, 137, 2167-2170, 1996).

Urocortin is another 40-amino-acid peptide related to urotensin I and CRF. cDNAs encoding urocortin from rat brain and human placenta have been analysed and peptides corresponding to putative mature rat and human urocortin synthesised. Synthetic rat or human urocortin binds to CRF-R1, CRF-R2α and CRF-R2β, and activates these receptors as measured by production of cAMP, its binding to and activation of the Type 2α and 2β receptors being much stronger than for CRF. (See J. Vaughan et al., Nature, 378, 287-292, 1995 (rat); C. J. Donaldson et al., Endocrinology, 137, 2167-2170, 1996 (human); WO 97/00063 (Salk Institute) (rat and human)).

WO 97/00063 suggests that urocortin or urocortin analogues could lower blood pressure, elevate mood, and improve memory and learning, and might possibly be administered to cause an improvement in short to medium term memory in a subject afflicted with Alzheimer's disease. (See also IDDB, entry Oct. 18, 1999 (Current Drugs Ltd) for Salk/Neurocrine Biosciences collaboration on urocortin; and Mar. 27, 2000 entry in R&D Insight (Adis International Ltd; accession number 13549) on Neurocrine Biosciences' development of small molecule mimetics of urocortin, which is mentioned as having a high affinity for the CRF2 receptor.) However, there is no disclosure or implicit or explicit suggestion in any of these last 3 documents that that urocortin inhibits neuronal cell death in patients of Alzheimer's or any other neurodegenerative disease, nor that the possible mechanism of action is via stimulation of type-1 CRF receptors. Rather the skilled reader is likely to think that, if any memory improvement is in fact achieved in Alzheimer's patients, then this is likely to be via enhancing existing memory paths e.g. by increasing neurotransmitter production by residual neurones in the Alzheimer's patient.

Cyclic CRF agonist peptides are disclosed in WO 98/54222 and WO 96/18649 (both Salk Institute) which are said to bind strongly to and activate CRF receptors. The WO 98/54222 peptides may be useful in lowering blood pressure, inflammation, the treatment of gastric ulcers and irritable bowel syndrome, and as diagnostics. The WO 96/18649 peptides are potentially indicated for modifying mood, learning, memory, behaviour, alertness, depression or anxiety, and for lowering blood pressure and inflammation. Linear peptides are disclosed in WO 85/03705 (Salk Institute) as CRF agonists for some of the above indications.

Various heterocyclic compounds have been made by Neurogen Corporation (see e.g. WO 98/21200, WO 98/45295, U.S. Pat. No. 5,723,608, WO 99/64422, WO 98/27066). These are suggested to be highly selective partial agonists or antagonists of human CRF 1 receptors with possible use for the treatment of stress-related disorders as well as depression, headache and anxiety.

A large number of publications exist decribing the synthesis and use of non-peptide small-molecule-heterocyclic compounds as CRF receptor antagonists, especially CRF receptor-1 antagonists, for various uses such as treatment of depression, anxiety, stress, substance abuse (for a review see J. R. McCarthy et al., Current Pharmaceutical Design, 5, 289-315, 1999 and P. J. Gilligan et al, J. Med. Chem., 43(9), 1641-1660, 2000, see pages 1650-1). Examples of publications include:

(1) WO 94/13676 (Pfizer, disclosing CRF receptor antagonists for the treatment of e.g. neurodegenerative diseases such as Alzheimer's disease) and D. W. Schultz et al., Proc. Natl. Acad. Sci., USA, 93, 10477, 1996 and Y. L. Chen et al., J. Med. Chem., 40, 1749-1754, 1997 disclosing CP154,526, a highly selective CRF receptor-1 antagonist;

(2) DuPont Merck workers publishing in WO 95/10506 (disclosing CRF receptor antagonists for the therapy of e.g. Alzheimer's disease), AG Arvanitis et al., J Med Chem, 42, 805-818, 1999 and CN Hodge et al., J Med Chem, 42, 819-832, 1999; and

(3) Taisho workers publishing in WO 98/42699 (=EP 0 976 745 A1), JP 11335373-A, JP 2000063277-A and JP 2000063378-A (all disclosing CRF receptor antagonists for the treatment of Alzheimer's disease, Parkinson's disease and Huntington's chorea) and also in S. Chaki et al., Eur. J. Pharmacol., 371, 205-211, 1999 and S. Okuyama et al., J. Pharmacol. Experimental Therapeut., 289(2), 926-935, 1999, the last two highlighting the potent and selective CRF receptor-1 antagonists CRA1000 and CRA1001.

### SUMMARY OF THE INVENTION

It is desirable to find further methods for treating central nervous system conditions or diseases, preferably by finding further classes of compounds which can be used in such treatments (e.g. including prophylaxis). It has now been discovered that CRF receptor agonists are useful to prevent or inhibit neuronal cell death in mammals suffering from or susceptible to certain nervous system diseases.

A first major aspect of the invention therefore provides the use of a CRF receptor agonist, or a pharmaceutically acceptable salt, complex or prodrug thereof, for the manufacture of a medicament for the prevention or inhibition of neuronal cell death in a mammal suffering from or susceptible to chronic neurodegenerative disease, traumatic (mechanical) neuronal injury, epilepsy-associated neuronal loss, paralysis, or spinal chord injury.

The present invention also provides a method of preventing or inhibiting neuronal cell death in a mammal suffering from or susceptible to chronic neurodegenerative disease, traumatic (mechanical) neuronal injury, epilepsy-associated neuronal loss, paralysis, or spinal chord injury, comprising administering to the mammal an effective amount of a CRF receptor agonist or a pharmaceutically acceptable salt, complex or prodrug thereof.

The invention also provides a CRF receptor agonist, or a pharmaceutically acceptable salt, complex or prodrug thereof, for use in the prevention or inhibition of neuronal cell death in a mammal suffering from or susceptible to chronic neurodegenerative disease, traumatic (mechanical) neuronal injury, epilepsy-associated neuronal loss, paralysis, or spinal chord injury.

This invention is unexpected due to some suggestions in the prior art that CRF and other CRF receptor agonists might be damaging to neurones or involved in neuronal damage, and other prior art such as WO 94/13676 (Pfizer), WO 95/10506 (Du Pont) and WO 98/42699 (=EP 0 976 745 A1), JP 11335373-A, JP 2000063277-A and JP 2000063378-A (all Taisho) which suggest that CRF receptor antagonists could be advantageously used in the treatment of such neurodegenerative diseases as Alzheimer's disease, Parkinson's disease or Huntington's chorea.

Compounds with CRF receptor agonist activity can be readily obtained by the skilled person. In particular, they can be identified by their ability to stimulate cAMP production (Battaglia, G., et al, Synapse (1987) 1:572-581). Neuronal cells, e.g. cerebellar granule neurons, or stably transfected cells containing the CRF receptors, e.g. transfected with CRF-R1 or other CRF receptor subtypes, can be subjected to putative CRF receptor ligands and intracellular cAMP can be measured with commercially-available cAMP enzyme immunoassay systems, e.g. as described in the Experimental Protocol section later, to determine activity. For stable transfection of cells, see: Rossant C J., et al, Endocrinology (1999) 140:1525-1536.

Preferably, CRF receptor agonists of the invention stimulate cAMP production more than 5 times compared to controls. Such criteria can optionally be used in a screen for selecting potential lead compounds having CRF receptor agonist activity.

Optionally, to confirm that cAMP production mediated by these test compounds occurs via stimulation of CRF receptors, compounds testing positive in the cAMP assay can be subjected to a second screen. In this second screen, cAMP production by the test compound can be measured both in the absence and presence of a non-selective CRF-receptor antagonist (i.e. which antagonises all CRF receptors or at least type-i, 2α and 2β receptors), e.g. by modifying Assay 4 herein accordingly. If cAMP production, and optionally also neuroprotection, mediated by the putative CRF receptor agonist under test is suppressed by the presence of the CRF receptor antagonist then this indicates CRF receptor agonist activity. Suitable CRF receptor antagonists for this purpose include astressin [available from Sigma (cat. no. A4933), see also J. Gulyas et al., Proc. Natl. Acad. Sci. USA, 92, p10575, 1995 and refs. cited therein]; compound 49 mentioned on page 1652 of P. J. Gilligan et al, J. Med. Chem., 43(9), 1641-1660, 2000 and described in U.S. Pat. No. 5,861,398 and D. R. Luthin et al., Bioorg. Med. Chem. Lett., 9, 765-770, 1999 (a combined CRF-R1 and CRF-R2 antagonist); and possibly the pyrimidine derivatives disclosed in EP 0976745 A1 (Taisho Pharmaceuticals).

Preferably, the medicament used, the method, or the agonist is for/of preventing or inhibiting apoptotic neuronal cell death.

Preferably, the mammal is suffering from or susceptible to chronic neurodegenerative disease, epilepsy-associated neuronal loss, paralysis or spinal chord injury. More preferably, the mammal is suffering from or susceptible to chronic neurodegenerative disease.

Chronic neurodegenerative diseases as defined herein include motor neurone disease or ALS, spongiform encephalopathy (e.g. bovine or Creutzfeldt-Jacob disease in humans), and, in humans, Alzheimer's disease, Parkinson's disease and Huntington's disease (chorea). ‘Chronic’ means or includes long continued; the opposite of acute. ‘Acute’ in disease refers to or includes symptoms, signs or course of intense character and of rapid onset, with early resolution in a certain direction e.g. convalescence, chronicity or mortality. ‘Neurodegenerative’ pertains to or is characterised by degeneration of nerve tissue. ‘Degeneration’ includes loss of cellular viability, loss of cellular function, and/or loss of cell number (neuronal or otherwise).

Preferably, the mammal is human. Preferably, the human is suffering from or susceptible to Alzheimer's disease, Parkinson's disease or Huntington's disease (chorea), most preferably Alzheimer's disease. See the supporting data in the Figures and Experimental Protocols section hereinafter, as well as the discussion on proteins linked to e.g. Alzheimer's disease under the third, fourth and fifth aspects of the invention below.

The mammal can be suffering from or susceptible to traumatic (mechanical) neuronal injury, for example traumatic (mechanical) brain or spinal chord injury.

CRF receptor agonists may also effect nerve repair or regeneration in the treatment of, for example, paralysis or spinal chord injury. ‘Nerve repair’ includes recovery of function. Lesions of the spinal chord can lead to loss of neurons by apoptosis as they no longer get their required growth factors, and CRF receptor agonists might be able to inhibit this apoptosis.

Therefore the second major aspect of the invention provides the use of a CRF receptor agonist, or a pharmaceutically acceptable salt, complex or prodrug thereof, for the manufacture of a medicament for the repair or regeneration of neuronal cells.

The invention also provides a method of repairing or regenerating neuronal cells in a mammal in need thereof, comprising administering to the mammal an effective amount of a CRF receptor agonist or a pharmaceutically acceptable salt, complex or prodrug thereof.

The invention also provides a CRF receptor agonist, or a pharmaceutically acceptable salt, complex or prodrug thereof, for use in the repair or regeneration of neuronal cells.

The medicament, method or agonist is preferably for the repair or regeneration of neuronal cells in a mammal (e.g. a human), more preferably in a mammal suffering from or susceptible to paralysis or spinal chord injury.

The third, fourth and fifth major aspects of the invention regard the pathway by which the neuronal cells die or survive. Approximately half of neurons die during development by a process called apoptosis, a programmed cell death with characteristic morphological and biochemical features, their survival being dependent at least partly on the availability of neurotrophic factors such as nerve growth factor (NGF) and insulin-like growth factors (IGF-1) (see R. W. Oppenheim, Annu. Rev. Neurosci., 14, 453-501, 1991; and E. M. Johnson and T. L. Deckworth, Annu. Rev. Neurosci., 16, 31-46, 1993; and references cited therein). Yao and Cooper (Science, 267, 2003-2006, 1995) discovered that the prevention of apoptosis by NGF requires the presence of an enzyme called phosphatidylinositol 3-kinase (phosphoinositide 3-kinase, PI 3-kinase or PI3K), a heterodimer of a 85 kDa regulatory subunit and a 110 kDa catalytic subunit (C L Carpenter et al., J. Biol. Chem., 296, 19704-19711, 1990; S J Morgan et al., Eur. J. Biochem., 191, 761-767, 1990; J. Escobedo et al., Cell, 65, 75-82, 1991; I. Hiles et al., Cell, 70, 419-429, 1992). Similarly, mildly depolarising concentrations of K⁺ (25 mM KCl) or the presence IGF-1 enables dissociated cerebellar granule cells to survive and leads to PI 3-kinase activation, whereas pharmacological inhibition of PI 3-kinase blocks the survival-promoting effects of K⁺ or IGF-1 leading to programmed cell death; these data suggest that PI 3-kinase activity is required for survival promotion by K⁺ or IGF-1 at least in vitro (T. M. Miller et al., J. Biol. Chem., 272, 9847-9853, 1997). However, PI 3-kinase inhibition had no effect on survival mediated by chlorophenylthio-cAMP (T. M. Miller et al., J. Biol. Chem., 272, 9847-9853, 1997). PI 3-kinase and Akt are necessary and sufficient for the survival of NGF-dependent sympathetic neurons, selective P13K inhibition by LY294002 causing cell death (R. J. Crowther and R. S. Freeman, J. Neurosci., 18, 2933-2943, 1998). Brain-derived neurotrophic factor (BDNF) also achieves motoneuron survival by signalling the PI3K pathway, as addition of LY 294002 at doses which inhibited Akt phosphorylation leads to abolition of the survival effects of BDNF (X. Dolcet et al., J. Neurochem., 73(2), 521-531, 1999).

Different P13K isoforms are described by Vanhaesebroeck et al. (Cancer Surveys 27, 249-270, 1996), including those which are activated by direct binding of Ras to the p110 catalytic subunit and those where G proteins activate forms of the enzyme which do not interact with the p85 regulatory subunit.

It is believed that activated PI 3-kinase activates another cellular protein called Akt (which has three isoforms Akt-1, -2 and -3) (Akt is sometimes also called protein kinase B), by means of direct binding of the phosphoinositide products of PI 3-kinase to the PH domain of Akt, translocation of Akt to the plasma membrane, and bi-phosphorylation of Akt at Ser⁴⁷³ and Thr³⁰⁸ by kinases (eg PDK1) themselves regulated by the phosphoinositide products of PI3K. Alternative PI3K-independent mechanisms of activation of Akt also exist. Activated Akt acts on several downstream cell components, eg phosphorylating and inhibiting the pro-apoptotic factors GSK3, BAD and caspase-9, and phosphorylating and activating IKK-α encouraging cell survival. Akt prevents cell death after withdrawal of growth factors or treatment of cells with apoptosis-inducers. See B. M. Marte, TIBS, Sep. 22, 1997, p355; T. F. Franke, Neural Notes, Vol V, issue 2, 3-7, 1999 and references cited therein for a review of Akt.

GSK-3 (glycogen synthase kinase-3) has two isoforms (α and β) sharing 85% amino-acid homology, both GSK-3α and GSK-3β showing good inter-species homology and both of which phosphorylate glycogen synthase (Embi et al., Eur. J. Biochem., 107, 519-527, 1980; J. R. Woodgett, Trends Biochem. Sci., 16, 177-181, 1991; J. R. Woodgett et al., Biochem. Soc. Trans., 21, 905-907, 1993; Cross et al., Biochem. J., 303, 21-26, 1994; G. I. Welsh et al., Trends Cell Biol., 6, 274-279, 1996; and refs cited therein). GSK-3 can be phosphorylated by and thereby inhibited by Akt, phosphorylation occurring at serine-21 of GSK-3α and serine-9 of GSK-3β (D.A.E. Cross, Nature, 378, 785-789, 1995). Phosphorylation of GSK-3α/β by other kinases also can occur (C. Sutherland, Febs Lett., 338, 37-42, 1994, and Biochem. J, 296, 15-19, 1993). Further, overexpression of catalytically active GSK-3 (GSK-3β) induces apoptosis in Rat-1 fibroblasts and neuronal-like PC12 cells, whereas dominant-negative mutant GSK-3 prevents apoptosis following inhibition of PI 3-kinase; the conclusion being that GSK-3 has an important role in the regulation of apoptosis and is an important downstream target of the PI 3-kinase/Akt cell-survival signalling pathway (M. Pap and G. M. Cooper, J. Biol. Chem., 273(32), 19929-19932, 1998). Similarly, by way of confirmation, trophic factor withdrawal or treatment with PI 3-kinase inhibitors in cultured cortical neurones led to stimulation of GSK3β activity preceding induction of apoptosis; and inhibiting or overexpressing GSK3β decreased or increased apoptosis respectively; the conclusion being that inhibition of GSK3β is one of the mechanisms by which PI 3-kinase activation protects neurones from programmed cell death (M. Hetman et al. J. Neurosci., Apr. 1, 2000, 20(7), 2567-2574).

BAD is a pro-apoptotic protein, which when phosphorylated by Akt leads to the phospho-BAD being bound by the 14-3-3 protein and thereby being less able to inhibit anti-apoptotic Bcl-2 molecules—see T. F. Gajewski et al., Cell, 87, 589, 1996, S. R. Datta et al., Cell, 91, 231-241, 1997, and refs cited therein. Similarly, the cell death protease caspase-9 is regulated by phosphorylation (M. H. Cardone et al., Science, 282, 1318-1321, 1998).

For reviews of the interaction of BAD and GSK-3 with Akt and/or PI3K, see B. M. Marte, TIBS, Sept. 22, 1997, p355; T. F. Franke, Neural Notes, Vol V, issue 2, 3-7, 1999.

Studies show that Akt is downregulated, GSK-3 affected and apoptosis induced by mutant PS1 (mutant presenilin-1, a cause of familial Alzheimer's disease), leading to the suggestion that downregulation of Akt may play a role in the pathogenesis of familial Alzheimer's disease (C. C. Weihl et al., J. Neurosci., 19, 5360-5369, 1999). Other authors suggest that the peptide amyloid β (a postulated contributor to neurodegeneration in Alzheimer's disease) inactivates PI3K, leading to activation of GSK-3β, tau phosphorylation and neuronal death (A. Takashima et al., Neuroscience Letters, 203, 33-66, 1996). Similarly, tau protein kinase I, whose homolog in rat brain is GSK-3β, is essential for amyloid β-protein-induced neurotoxicity and was linked to Alzheimer's disease (A. Takashima et al., Proc. Natl. Acad. Sci USA, 90, 7789-7793, 1993, see p. 7789 and conclusion on p. 7792). There are also a number of papers showing that GSK-3 phosphorylates tau, hyperphosphorylation of which might be a cause of Alzheimer's disease, the papers thereby linking GSK-3 activity with Alzheimer's disease (M. Hong and V. M. -Y. Lee, J. Biol. Chem., 272(31), 19547-19553, 1997 and references 14-16, 21 and 22 cited therein). Further, there are several publications disclosing small-molecule GSK-3 inhibitors for the treatment of various diseases, in particular chronic neurodegenerative diseases including dementias such as Alzheimer's disease—see e.g. WO 00/21927 A2 and A3, WO 00/38675 and WO 01/09106 A1, all in the name of SmithKline Beecham plc, and WO 98/16528 in the name of Chiron.

It is desirable to find classes of compounds which have an effect on certain biochemical events and/or pathways, for example apoptosis and/or the PI 3-kinase signalling pathway. It has now been discovered that CRF receptor agonists protect (rescue) neurones such as cerebellar granule neurones from apoptosis caused by PI 3-kinase signalling pathway inhibition, as shown by the results presented in the Figures and in the Experimental Protocols section hereinafter.

Therefore, a third major aspect of the invention provides the use of a CRF receptor agonist, or a pharmaceutically acceptable salt, complex or prodrug thereof, for the manufacture of a medicament for the prevention or inhibition of apoptotic neuronal cell death, for example in a mammal (e.g. human).

The invention also provides a method of preventing or inhibiting apoptotic neuronal cell death in a mammal, comprising administering to the mammal an effective amount of a CRF receptor agonist, or a pharmaceutically acceptable salt, complex or prodrug thereof.

The invention also provides a CRF receptor agonist, or a pharmaceutically acceptable salt, complex or prodrug thereof, for use in the prevention or inhibition of apoptotic neuronal cell death, for example in a mammal (e.g. human).

Apoptosis or apoptotic, as defined herein, refers to a programmed cell death with characteristic morphological and biochemical features known to those skilled in the art (see for example R. W. Oppenheim, Annu. Rev. Neurosci., 14, 453-501, 1991; and E. M. Johnson and T. L. Deckworth, Annu. Rev. Neurosci., 16, 31-46, 1993; and references cited therein).

A fourth major aspect of the present invention provides the use of a CRF receptor agonist, or a pharmaceutically acceptable salt, complex or prodrug thereof, for the manufacture of a medicament for the prevention or inhibition of neuronal cell death potentiated by inhibition or suppression of the PI 3-kinase signalling pathway.

The invention also provides a method of preventing or inhibiting neuronal cell death in a mammal, the cell death being potentiated by inhibition or suppression of the PI 3-kinase signalling pathway, comprising administering to the mammal an effective amount of a CRF receptor agonist, or a pharmaceutically acceptable salt, complex or prodrug thereof.

The invention also provides a CRF receptor agonist, or a pharmaceutically acceptable salt, complex or prodrug thereof, for use in the prevention or inhibition of neuronal cell death potentiated by inhibition or suppression of the PI 3-kinase signalling pathway.

The PI 3-kinase signalling pathway as defined herein refers to the pathway by which activated PI 3-kinase suppresses neuronal cell death (e.g. by apoptosis). This pathway includes:

(i) PI 3-kinase itself (see for example: C L Carpenter et al., J. Biol. Chem., 296, 19704-19711, 1990; S J Morgan et al., Eur. J. Biochem., 191, 761-767, 1990; J. Escobedo et al., Cell, 65, 75-82, 1991; I. Hiles et al., Cell, 70, 419-429, 1992; and Vanhaesebroeck et al. Cancer Surveys 27, 249-270, 1996) including various isoforms of P13K (see e.g. Vanhaesebroeck et al. Cancer Surveys 27, 249-270, 1996);

(ii) Akt (especially Akt-1 but also Akt-2 and Akt-3 and other isoforms); and other proteins which both (a) act to promote cell survival or inhibit cell death e.g. apoptotic cell death and (b) are expressed, activated (e.g. by phosphorylation), de-inhibited and/or reactivated in response to signals generated by PI3K or in a manner dependent on PI3K;

(iii) the signals emitted by PI3K which activate Akt (e.g. Akt-1) and/or PDK1, these signals including phosphoinositides such as phosphatidylinositol (3,4,5)-triphosphate [PtdIns(3,4,5)P₃] and phosphatidylinositol (3,4)-bisphosphate [PtdIns(3,4)P₂];

(iv) kinases which are themselves regulated by the phosphoinositide products of PI3K and which have a role in cell survival (e.g. by activation of Akt or similar cell-survival proteins), these kinases including PDK1 (PtdIns(3,4,5)P₃-dependent kinase 1; see e.g. D. R. Alessi et al., Curr. Biol., 7, 261-269, 1997 and C. Belham et al., Curr. Biol., 9, R93, 1999 and refs cited therein);

(v) translocation of Akt and similar cell-survival proteins to the plasma membrane;

(vi) activation, for example by phosphorylation, of Akt and similar cell-survival proteins;

(vii) the promotive action (e.g. activation, decreased inhibition and/or reactivation, e.g. by phosphorylation) of Akt and similar cell-survival proteins on downstream proteins which promote or are involved in cell survival, such as IKK-α; and

(viii) the suppression (e.g. reduced activation, inhibition and/or deactivation, e.g. by phosphorylation), e.g. by Akt and similar cell-survival proteins, of downstream proteins which promote cell death/apoptosis; these downstream proteins including GSK-3, caspase-9 and BAD.

For the interaction of the proteins in (vii) and (viii) above such as GSK-3 and BAD with Akt or PI3K, and for Akt and PI3K in general, see B. M. Marte, TIBS, Sept. 22, 1997, p355; T. F. Franke, Neural Notes, Vol V, issue 2, 3-7, 1999 (reviews) and references cited therein and/or the references referred to above.

In a disease and/or condition against which the CRF receptor agonists can be used, the neuronal cell death can for example be potentiated by reduced expression, reduced activation, inhibition and/or deactivation of PI 3-kinase present in the neuronal cells. Alternatively or additionally, in a disease and/or condition, the neuronal cell death can be potentiated by reduced expression, reduced activation, inhibition and/or deactivation of Akt (e.g. Akt-1) present in the neuronal cells. Alternatively or additionally, in a disease and/or condition, the neuronal cell death can be potentiated by activation of a cell-death/apoptosis-promoting protein downstream of Akt, preferably GSK-3, more preferably GSK-3β, present in the neuronal cells.

Alternatively or additionally, in some diseases or conditions, one or more of the components (i) to (viii) of the PI 3-kinase signalling pathway as defined above can be inhibited or suppressed.

The fourth and forthcoming fifth aspects of the invention are supported by the evidence in the Experimental Protocol section and Figures hereinafter in which, inter alia, CRF receptor agonists are found to confer at least partial protection against neuronal cell death caused by selective inhibition of PI 3-kinase by LY 294002, this protection seemingly being mediated at least in part by indirect interaction of the CRF receptor agonists with GSK-3 on the PI 3-kinase pathway.

A fifth major aspect of the present invention provides the use of a CRF receptor agonist, or a pharmaceutically acceptable salt, complex or prodrug thereof, for the manufacture of a medicament for preventing or inhibiting neuronal cell death by stimulating or activating the PI 3-kinase signalling pathway.

The invention also provides a method of preventing or inhibiting neuronal cell death in a mammal by stimulating or activating the PI 3-kinase signalling pathway, comprising administering to the mammal an effective amount of a CRF receptor agonist, or a pharmaceutically acceptable salt, complex or prodrug thereof.

The invention also provides a CRF receptor agonist, or a pharmaceutically acceptable salt, complex or prodrug thereof, for use in the prevention or inhibition of neuronal cell death by stimulating or activating the PI 3-kinase signalling pathway.

In the invention, the PI 3-kinase signalling pathway can be stimulated or activated by increased expression, increased activation, decreased inhibition and/or reactivation of PI 3-kinase present in the neuronal cells. Alternatively or additionally, the PI 3-kinase signalling pathway can be stimulated or activated by increased expression, increased activation, decreased inhibition and/or reactivation of Akt (e.g. Akt-1) present in the neuronal cells. Alternatively or additionally, the PI 3-kinase signalling pathway can be stimulated or activated by suppression (e.g. reduced activation, inhibition and/or deactivation, e.g. by phosphorylation) of one or more cell-death/apoptosis-promoting proteins downstream of Akt present in the neuronal cells. Alternatively or additionally, it is preferable that the PI 3-kinase signalling pathway is stimulated or activated at least in part by suppression (e.g. reduced activation, inhibition and/or deactivation, in particular by phosphorylation) of GSK-3, more preferably GSK-3β (e.g. by phosphorylation at serine-9), present in the neuronal cells. Alternatively or additionally, one or more of the components (i) to (viii) of the PI 3-kinase signalling pathway as defined above can be stimulated or activated.

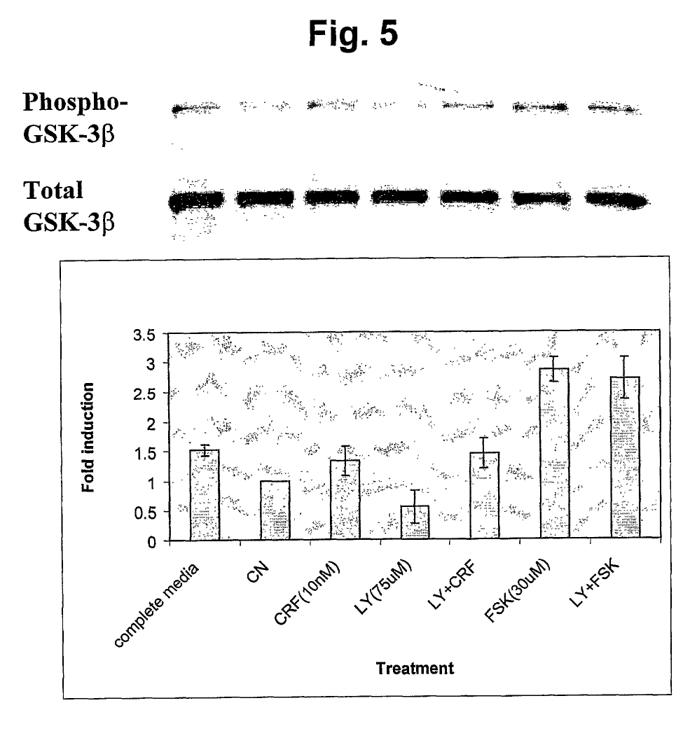

Also provided is the use of a CRF receptor agonist, or a pharmaceutically acceptable salt, complex or prodrug thereof, for the manufacture of a medicament for preventing or inhibiting neuronal cell death (e.g. at least in part) by suppression of GSK-3 (e.g. GSK-3β) present in the neuronal cells. Preferably the GSK-3 is suppressed by inhibition, in particular by phosphorylation. Also provided is a method of preventing or inhibiting neuronal cell death in a mammal (e.g. at least in part) by suppression of GSK-3 (e.g. GSK-3β) present in the neuronal cells, comprising administering to the mammal an effective amount of a CRF receptor agonist, or a pharmaceutically acceptable salt, complex or prodrug thereof. See discussion on the results presented in FIG. 5 hereinafter which supports this.

In the fourth and fifth aspects of the invention, the neuronal cell death can be in a mammal (e.g. human).

In the fourth, fifth and other aspects of the invention, the medicament used, the method, or the agonist is preferably for/of preventing or inhibiting apoptotic neuronal cell death.

In all aspects of the invention, the medicament used, the method, or the agonist is preferably for/of preventing or inhibiting neuronal cell death in the central nervous system (CNS), in particular for/of preventing or inhibiting cerebral neuronal cell death (e.g. in the cortex, hippocampus, striatum and/or hypothalamus).

In the third, fourth, fifth and other aspects of the invention, the prevention or inhibition of neuronal cell death is preferably potentiated by increasing the levels of intracellular cyclic adenosine monophosphate (cAMP) in the neuronal cells.

Cyclic AMP is involved in the cardiovascular and the nervous system, in immune mechanisms, in cell growth and differentiation, and in general metabolism. Moreover, cyclic AMP elevation by drugs (e.g. forskolin) which directly stimulate its synthesis can protect cerebellar granule neurones from apoptotic death resulting from a lack of growth signal (S. R D'Mello et al., Proc. Natl. Acad. Sci. USA 90, 10989-10993, 1993).

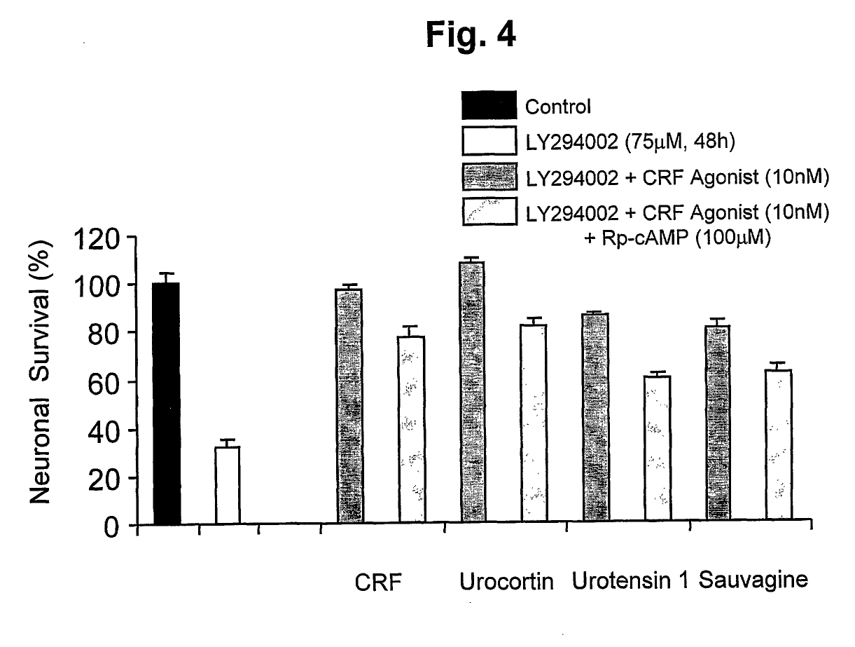

It is believed that CRF receptor agonists at least partially exert their rescuing effect by stimulating cAMP production. This is because in the tests conducted (see later—FIG. 4), treatment with CRF receptor agonists leads to potent stimulation of cAMP, but the neuroprotective effects of these agonists were partially antagonised when an inhibitor of cAMP (Rp-cAMP, an isomer of cAMP—see Gjertsen B T et al, J. Biol. Chem (1995) 270:20599-20604) was used. Without intending to be bound by theory, cAMP might interact directly or indirectly with one or more positions/aspects of the PI 3-kinase signalling pathway. For example, the results shown in FIG. 5 hereinafter suggest possible interaction with GSK-3. However, the failure of Rp-cAMP in FIG. 4 to completely suppress CRF's protective activity suggests that another (as yet unknown) messenger system other than cAMP is mediating the protective effects of CRF receptor stimulation.

In the third, fourth, fifth and other aspects of the invention, the medicament, method or agonist is preferably for the prevention or inhibition of neuronal cell death in a mammal, e.g. a human, especially a mammal suffering from or susceptible to chronic neurodegenerative disease, traumatic (mechanical) neuronal injury, epilepsy-associated neuronal loss, paralysis or spinal chord injury. The medicament, method or agonist is more preferably for the prevention or inhibition of neuronal cell death in a human suffering from or susceptible to Alzheimer's disease, Parkinson's disease or Huntington's disease, in particular Alzheimer's disease

In all aspects of the invention, the CRF receptor agonist is preferably a CRF receptor-1 agonist (CRF receptor-1 being defined hereinabove), in which case preferably the neuronal cell death is prevented or inhibited, or the neuronal cells are repaired or regenerated, by stimulating CRF receptor-1 (which does not exclude the possibility that additional neuroprotective mechanisms may be acting). It is thought that the CRF receptor agonists mainly (or at least partly) exert their neuroprotective effect by stimulating CR-F receptor-1, judging by the fact that addition of the selective CRF-R1 antagonist CP154,526 blocks the neuroprotective effect of CRF (see tests later). The general tests given either directly below, or the more specific Assays 1-6 along with the “Use of the Assays . . .” section given in the Experimental Protocol section hereinafter, allow determination of whether neuroprotection is mediated by stimulation of a CRF receptor such as CRF receptor-1.

More preferably, in all aspects of the invention, the CRF receptor agonist is a selective CRF receptor-1 agonist, i.e. binds to and/or stimulates the CRF receptor-1 at least five times as strongly as it does CRF receptor-2 (e.g. CRF receptors-2α and/or -2β). Still more preferably, the CRF receptor agonist is a selective CRF receptor-1 agonist which binds to (even more preferably binds to and stimulates) the CRF receptor-1 at least five times as strongly as it does CRF receptor-2 (e.g. CRF receptors-2α and/or -2β). In all cases, the selectivity is preferably measured with respect to human CRF receptors. CRF is such a CRF-R1 selective ligand (rat/human CRF binds to CRF-R1/2α/2β at 0.95/13/17 nM and accumulates cAMP in stably transfected CHO cells expressing CRF-R1/2α/2β at EC₅₀ values of 0.26/5.3/3.0 nM—see C. J. Donaldson et al., Endocrinology, 137, 2167-2170, 1996 and J. Vaughan et al., Nature, 378, 287-292, 1995). The CRF receptor agonist preferably should not significantly activate ACTH receptors or glucocortoid (steroid) receptors, i.e. is selective for activation of CRF receptor(s), e.g. CRF receptor-1, over these receptors.

To measure CRF receptor-1 agonist activity, cells stably transfected with CRF-R1 (e.g. see R. Chen et al., Proc. Natl. Acad. Sci. USA, 90, 8967-8971, 1993; J. Vaughan et al., Nature, 378, 287-292, 1995, Table 1 and references cited in these two articles) can be subjected to the putative CRF receptor ligands and intracellular cAMP production can be measured (e.g. as described in a modified Assay 3 herein) as a measure of CRF-R1 stimulation. To measure selectivity, especially selectivity of stimulation of -1 compared to -2 receptors, this would be followed by cross-screens with cells stably transfected with CRF-R2α and/or R2β (e.g. see WO 95/34651, pages 43 to 48; for R2α transfected into CHO-pro5 cells see N. Suman-Chauchan et al., Eur. J. Pharmacol., 379, 1999, 219-227, section 2), again using cAMP as a measure of stimulation of those receptors. To confirm that the cAMP production is primarily caused by stimulation of CRF receptor-1, cAMP production by the test compound should preferably be measured both in the absence and presence of a selective CRF-R1 antagonist such as CP154,526 (e.g. by modifying Assay 4 hereinafter)—if cAMP production, and optionally also neuroprotection, mediated by the CRF receptor agonist is suppressed by the presence of a selective CRF-R1 antagonist then this indicates CRF receptor-1 agonist activity. CP154,526 is disclosed in WO 94/13676; D. W. Schultz et al., Proc. Natl. Acad. Sci., USA, 93, 10477, 1996; Y. L. Chen et al., J. Med. Chem., 40, 1749-1754, 1997; and is reviewed in J. R. McCarthy et al., Current Pharmaceutical Design, 5, 289-315, 1999.

Selectivity of binding (affinity) to CRF-R1 compared to CRF-R2 can also be measured using conventional radioligand binding-competitive displacement techniques using each of the receptors to be compared, such techniques for example being described in: N. Suman-Chauchan et al., Eur. J. Pharmacol., 379, 1999, 219-227 (see e.g. section 2.7-[¹²⁵I][tyr⁰]sauvagine binding to rat or human CRF-R1 or CRF-R2α); D. E. Grigoriadis et al., Mol. Pharmacol., 50, 1996, 679; R. Chen et al., Proc. Natl. Acad. Sci. USA, 90, 8967-8971, 1993 (see materials and methods and eg FIG. 3) and M H Perrin et al., Endocrinology, 118, 1986, 1171-1179.

Alternatively, to screen test compounds for CRF receptor-1 agonist activity, cAMP production mediated by the test compounds can be measured in cerebellar granule neurones or similar cells (see e.g. Assay 3 below). The test compounds stimulating cAMP production by more than a threshold multiplier, e.g. 5 times, compared to controls can be selected for a second screen. In the second screen, cAMP production by the test compound is measured in the same type of cells in the presence of a selective CRF-R1 antagonist such as CP154,526 (see e.g. Assay 4 below)—again, if cAMP production mediated by the CRF receptor agonist is suppressed by the presence of a selective CRF-R1 antagonist then this indicates CRF receptor-1 agonist activity. An optional third screen (see e.g. Assay 2 below) would be to compare the neuroprotection conferred by the CRF receptor agonist with that conferred by the agonist in the presence one or more concentrations of the CRF-R1 antagonist—a decrease in neuroprotection here indicates that neuroprotection by the test compound is mediated via stimulation of CRF-R1.

Measuring CRF receptor binding could be useful as a secondary screen, in which case this can be done by known methods (see e.g. WO 95/34651, page 45, EP 0976745A1 pages 19-20, WO 98/45295 pages 15-16, R. Chen et al., Proc. Natl. Acad. Sci. USA, 90, 8967-8971, 1993; J. Vaughan et al., Nature, 378, 287-292, 1995, Table 1 and relevant references cited in these publications).

Optionally, the CRF receptor-1 agonist has an Eₘₐₓ value of 50% or more at CRF receptor-1 measured relative to CRF as a standard. The Eₘₐₓ value represents the maximum efficacy compared empirically to CRF as the full agonist of choice, i.e. Eₘₐₓ=the maximum response of CRF receptor-1 in a defined system to the agonist under test as a percentage of the maximum response of the same system to CRF under the same conditions. See e.g. D. Smart et al., Eur. J. Pharmacol., 379, 1999, 229-235 and N. Suman-Chauchan et al., ibid, 219-227 for one possible response measurement method (the Cytosensor microphysiometer which measures extracellular acidification rate can be replaced by other standard e.g. cAMP measurements) and CHO-pro5 cell culture system. Therefore, partial agonists with an Eₘₐₓ value of less than 50% at CRF receptor-1 measured relative to CRF as a standard may not be preferred. Optionally, the CRF receptor-1 agonists have an E-max greater than or equal to 75%, still more preferably greater than or equal to 90%, relative to CRF. The agonist can be a full agonist, i.e. having substantially the same maximum efficacy as CRF (i.e. Eₘₐₓ=about 100% cf. CRF).

Optionally, the CRF receptor agonist, has substantially no or minimal antagonist activity at any CRF receptor (so for example is not both a CRF receptor-1 agonist and a CRF receptor-2 antagonist).

In all aspects of the invention, the CRF receptor agonist or CRF receptor-1 agonist optionally comprises CRF, urocortin, sauvagine or urotensin 1, or a pharmaceutically acceptable salt, complex or prodrug thereof. These compounds are described in references cited above, and are shown to be effective in protecting cerebellar granule neurones from death caused by PI 3-kinase inhibition in the tests presented below.

In all aspects of the invention, the use/method/agonist/medicament can involve delayed administration to a/the mammal of (e.g. an effective amount of) a CRF receptor agonist (e.g. CRF receptor-1 agonist), or a pharmaceutically acceptable salt, complex or prodrug thereof, after an acute neurodegenerative or potentially neurodegenerative occurrence (e.g. traumatic/mechanical neuronal injury or cerebral ischaemia/stroke). The time of administration can be 30 or 60 minutes or more after the said occurrence, and/or can be up to 8 or 6 or 4 or 2 or 1 hour(s) after the said occurrence, e.g. 30 mins to 8 hours, 30 mins to 6 hours, or 30 mins to 4 hours after said occurrence. CRF receptor agonists might be neuroprotective when administered within these time frames after such occurrences (see FIGS. 5A and 5B later), which would allow administration in hospital after the occurrence.

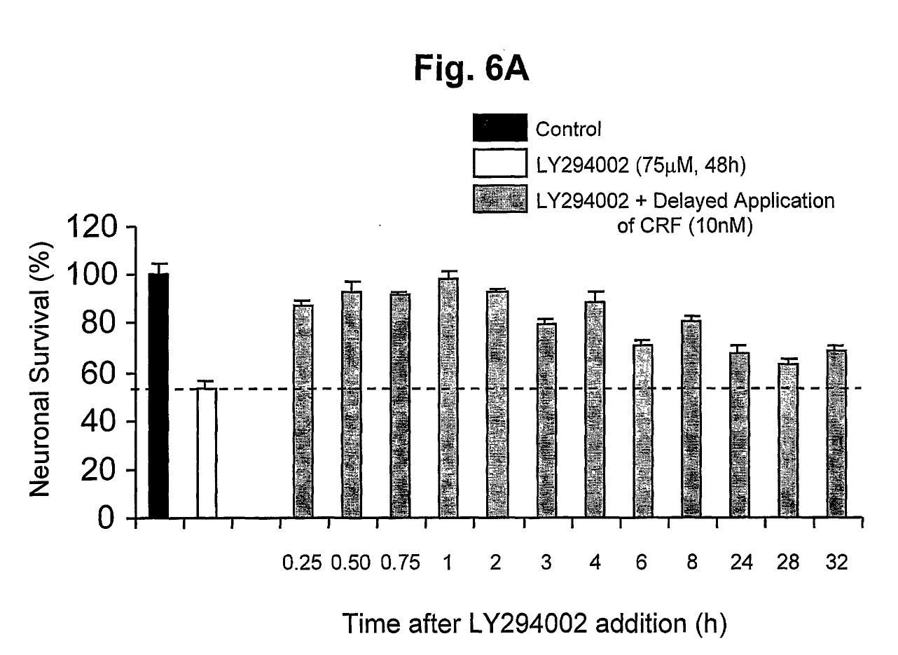

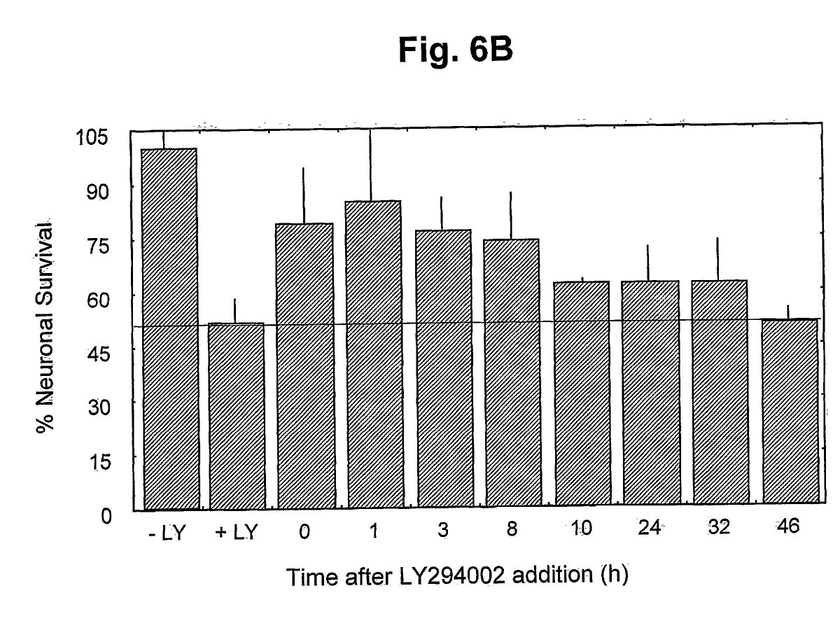

It has also been discovered the CRF receptor-1 agonists are useful to prevent or inhibit neuronal cell death in mammals suffering from or susceptible to cerebral ischaemia (stroke) (see results from the in vivo cerebral ischaemia model shown in FIG. 6 hereinafter).

A sixth major aspect of the invention therefore provides the use of a CRF receptor-1 agonist, or a pharmaceutically acceptable salt, complex or prodrug thereof, for the manufacture of a medicament for preventing or inhibiting neuronal cell death, in a mammal suffering from or susceptible to cerebral ischaemia, by stimulating type-1 CRF receptors (CRF receptor-1).

The invention also provides a method of preventing or inhibiting neuronal cell death in a mammal suffering from or suceptible to cerebral ischaemia, comprising stimulating type-1 CRF receptors (CRF receptor-1) in the mammal by administering to the mammal an effective amount of a CRF receptor-1 agonist, or a pharmaceutically acceptable salt, complex or prodrug thereof.

The invention also provides a CRF receptor-1 agonist, or a pharmaceutically acceptable salt, complex or prodrug thereof, for use in preventing or inhibiting neuronal cell death, in a mammal suffering from or susceptible to cerebral ischaemia, by stimulating type-1 CRF receptors (CRF receptor-1).

Therefore, in the third, fourth, fifth aspects of the invention, the medicament, method or agonist can also be for the prevention or inhibition of neuronal cell death in a mammal, e.g. a human, suffering from or susceptible to cerebral ischaemia.

This sixth aspect of the invention, which is supported by results from the in vivo cerebral ischaemia (stroke) model shown in FIG. 6 hereinafter, is unexpected due to the suggestions in the prior art that CRF and other CRF receptor agonists might be damaging to neurones or might mediate neuronal damage during cerebral ischaemia (see e.g. M. Lyons et al., Brain Res., 545, 339-342, 1991 and P. J. L. M. Strijbos et al., Brain Res., 656, 405-408, 1994). As discussed above, the general CRF-R1 tests given above or the specific Assays 2, 3, 4 and/or 5 given in the Experimental Protocol section hereinafter, can be used to determine whether a given compound mediates neuroprotection by stimulation of CRF receptor-1.

Formulation and Dosing

In order to use CRF receptor agonists in therapy, they will normally be formulated into a pharmaceutical composition in accordance with standard pharmaceutical practice.

CRF receptor agonists may conveniently be administered by any of the routes conventionally used for drug administration, for instance, parenterally, orally, topically or by inhalation. CRF receptor agonists may be administered in conventional dosage forms prepared by combining then with standard pharmaceutical carriers according to conventional procedures. CRF receptor agonists may also be administered in conventional dosages in combination with a known, second therapeutically active compound. These procedures may involve mixing, granulating and compressing or dissolving the ingredients as appropriate to the desired preparation. It will be appreciated that the form and character of the pharmaceutically acceptable carrier is dictated by the amount of active ingredient with which it is to be combined, the route of administration and other well-known variables. The carrier(s) must be “acceptable” in the sense of being compatible with the other ingredients of the formulation and not deleterious to the recipient thereof.

The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time delay material well known to the art, such as glyceryl mono-stearate or glyceryl distearate alone or with a wax.

A wide variety of pharmaceutical forms can be employed. Thus, if a solid carrier is used, the preparation can be tableted, placed in a hard gelatin capsule in powder or pellet form or in the form of a troche or lozenge. The amount of solid carrier will vary widely but preferably will be from about 25 mg to about 1 g. When a liquid carrier is used, the preparation will be in the form of a syrup, emulsion, soft gelatin capsule, sterile injectable liquid such as an ampoule or nonaqueous liquid suspension.

CRF receptor agonists are preferably administered parenterally, that is by intravenous, intramuscular, subcutaneous intranasal, intrarectal, intravaginal or intraperitoneal administration. The intravenous form of parenteral administration is generally preferred. Appropriate dosage forms for such administration may be prepared by conventional techniques.

CRF receptor agonists may also be administered orally. Appropriate dosage forms for such administration may be prepared by conventional techniques.

CRF receptor agonists may also be administered by inhalation, that is by intranasal and oral inhalation administration. Appropriate dosage forms for such administration, such as aerosol formulations, may be prepared by conventional techniques.

CRF receptor agonists may also be administered topically, that is by non-systemic administration. This includes the application of the CRF receptor agonists externally to the epidermis or the buccal cavity and the instillation of such a compound into the ear, eye and nose, such that the compound does not significantly enter the blood stream.

For all methods of use disclosed herein for CRF agonists, especially undisclosed small-molecule agonists, the daily oral dosage regimen can optionally be from about 0.1 to about 80 mg/kg of total body weight, preferably from about 0.2 to 30 mg/kg, more preferably from about 0.5 mg to 15 mg/kg. The daily parenteral dosage regimen can optionally be about 0.1 to about 80 mg/kg of total body weight, preferably from about 0.2 to about 30 mg/kg, and more preferably from about 0.5 mg to 15 mg/kg. The daily topical dosage regimen can optionally be from 0.1 mg to 150 mg/kg, administered one to four, preferably two or three times daily. The daily inhalation dosage regimen can optionally be from about 0.01 mg/kg to about 1 mg/kg per day. However, for peptide agonists such as CRF, urotensin, urocortin, etc., much lower dosages may be appropriate (U.S. Pat. No. 4,489,163 says in vivo doses in rats of from 30 ng to 3 μg of rCRF per kg body weight rapidly elevated ACTH and β-endorphin-like secretion; whereas Behan in Nature, 378, 1995, p284 at page 286 uses 0.1 to 25 μg CRF per rat (see FIG. 3) to test memory and anxiety in rats). As suggested beforehand, it is preferred to administer doses of CRF agonists that do not substantially stimulate ACTH, β-endorphin or corticosteroid production/release.

It will also be recognized by one of skill in the art that the optimal quantity and spacing of individual dosages of the inhibitors will be determined by the nature and extent of the condition being treated, the form, route and site of administration, and the particular patient being treated, and that such optimums can be determined by conventional techniques. It will also be appreciated by one of sill in the art that the optimal course of treatment, i.e., the number of doses of the CRF receptor agonists given per day for a defined number of days, can be ascertained by those skilled in the art using conventional course of treatment determination tests.

An advantageous buffered liquid formulation for the CRF peptide is disclosed in WO 98/11912 comprising CRF, a buffer to maintain the pH in the range of 2-5 or 6-9 when in liquid form and an alcohol such as mannitol, sorbitol, methanol, glycerol or the like This is stated to confer improved stability during long-term storage as a liquid. Such a formulation might also be advantageous for agonist peptides similar to CRF.

All publications, including but not limited to patents and patent applications, cited in this specification are herein incorporated by reference as if each individual publication were specifically and individually indicated to be incorporated by reference herein as though fully set forth.

### EXAMPLES AND EXPERIMENTAL PROTOCOLS

The invention will now be described by reference to the following examples which are merely illustrative and are not to be construed as a limitation of the scope of the present invention. Some of the examples are described with reference to the figures in which:

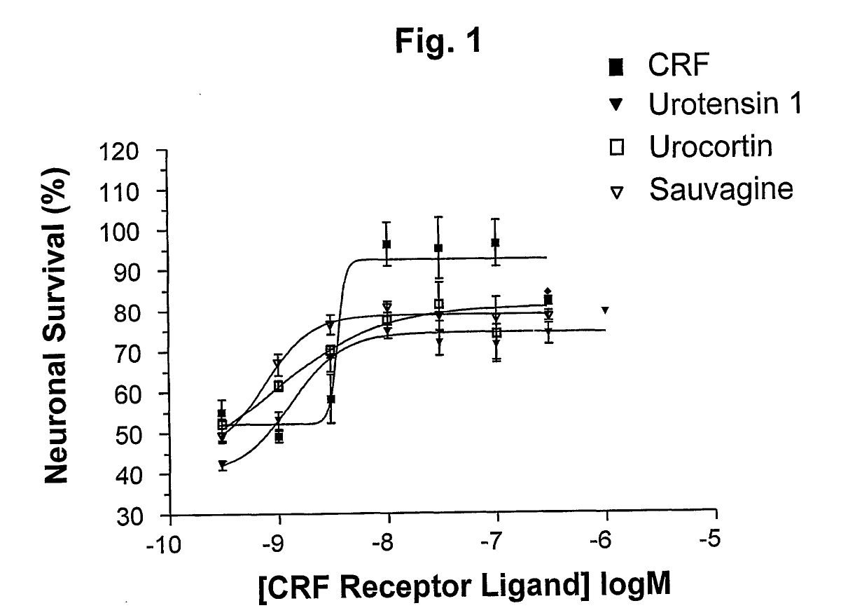

FIG. 1 is a graph illustrating percentage mean survival of cerebellar granule neurones, when in the presence of the PI 3-kinase inhibitor LY 294002 and also a CRF receptor agonist (CRF, urocortin, urotensin 1, or sauvagine), as a function of agonist concentration;

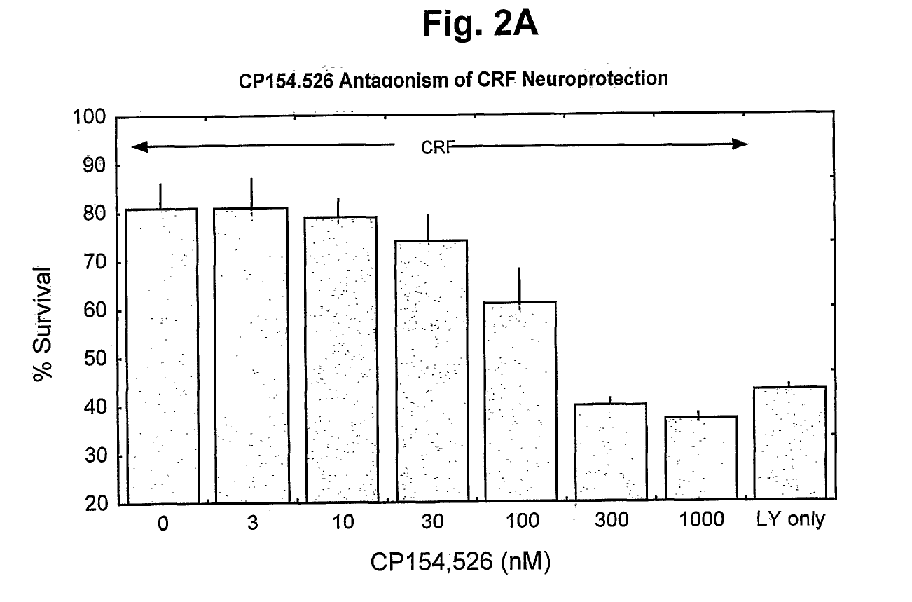

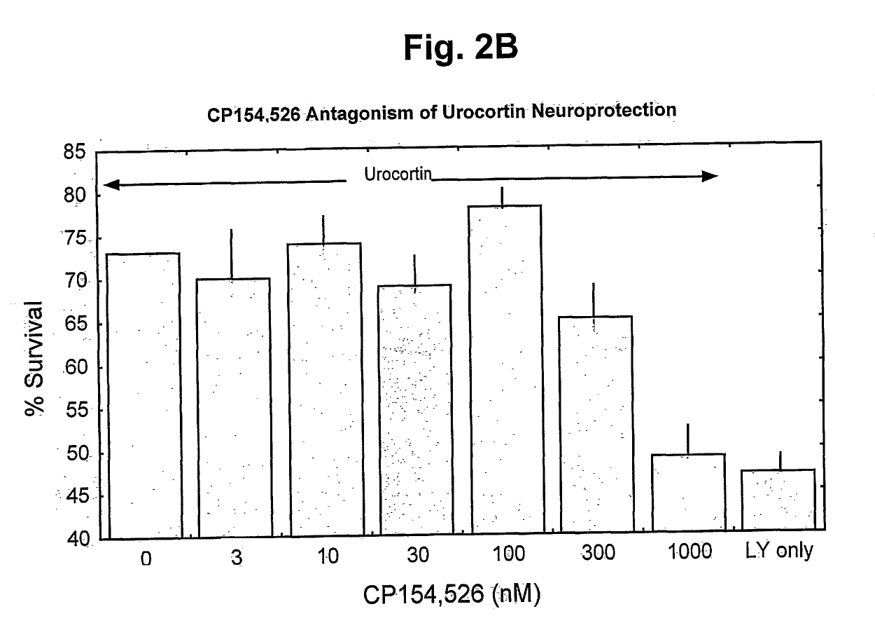

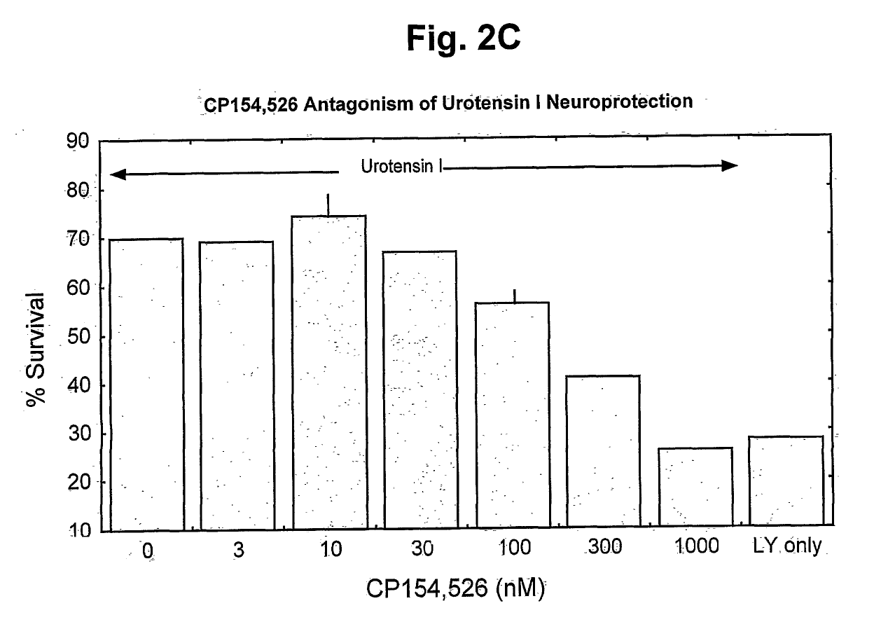

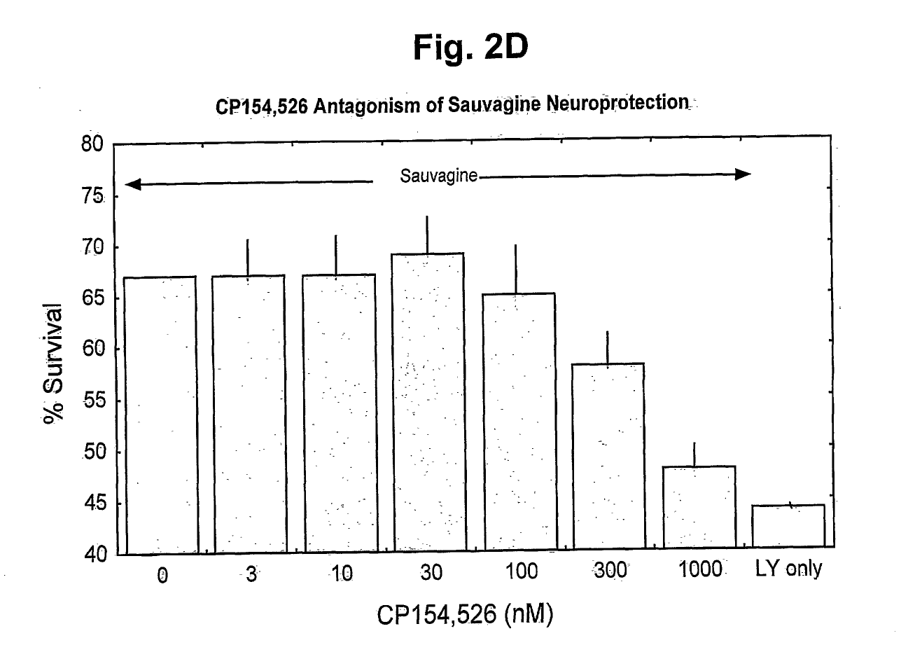

FIGS. 2A-D are bar graphs illustrating the effects of CP154,526, a selective CRF receptor-1 antagonist, on the protective effects of (A) CRF, (B) urocortin, (C) urotensin I, and (D) sauvagine against neurotoxicity induced by LY294002 in primary cerebellar granule neurones;

FIG. 3 is a graph illustrating cAMP synthesis induced by CRF receptor agonists, measured in absolute values of cAMP per cell number, in primary cerebellar granule neurones, as a function of agonist concentration;

FIG. 4 is a bar graph illustrating percentage mean survival of primary cerebellar granule neurones, when in the presence of the PI 3-kinase inhibitor LY 294002 (75 μM) and also a CRF receptor agonist at 10 μM (CRF, urocortin, urotensin 1, or sauvagine), in the absence or presence of the cAMP inhibitor Rp-cAMP (100 μM);

FIG. 5 is a bar graph and superimposed Western blot electrophoresis gel showing levels of serine-9-phosphorylated GSK-3β (phospho-GSK-3β) and total GSK-3β in cerebellar granule neurones cells in the presence of (from right to left) complete medium, control serum-free medium (CN), CRF, LY 294002, CRF+LY 294002, forskolin (FSK), and LY 294002+FSK.

FIGS. 6A and 6B are bar graphs illustrating percentage mean survival of primary cerebellar granule neurones, when in the presence of the PI 3-kinase inhibitor LY 294002 (75 μM) and CRF (10 nM) added at the same time as LY 294002 or at different times (shown in hours) following LY 294002 addition, the results showing that delayed CRF addition is sufficient to protect cerebellar granule neurons from injury by LY 294002; and

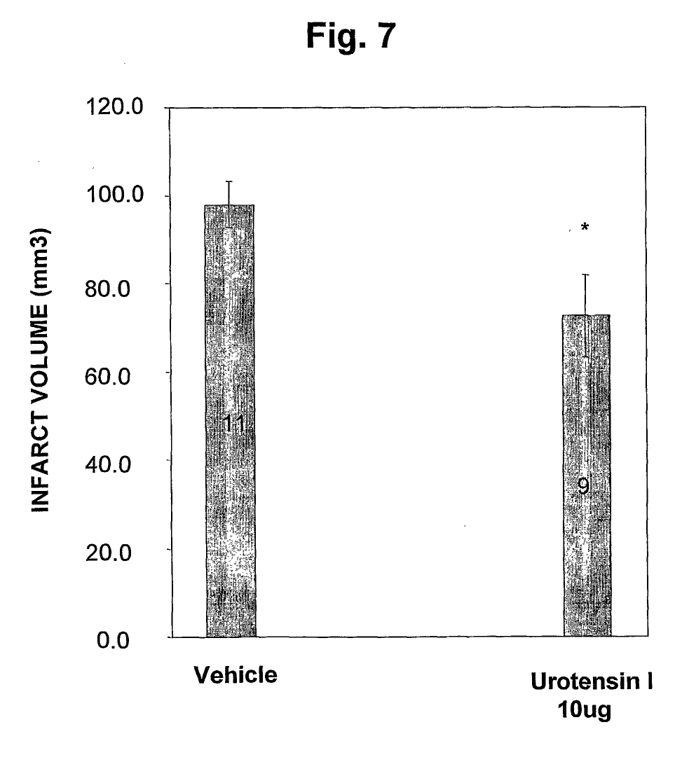

FIG. 7 is a bar graph showing the effects of administration of intracerebroventricular (icv) urotensin I (10 μg/rat) following distal middle cerebral artery occlusion (MCAO) in spontaneous hypertensive rats (SHR), as measured by infarct volume (mm³) and a numerical scoring system for neurological deficits.

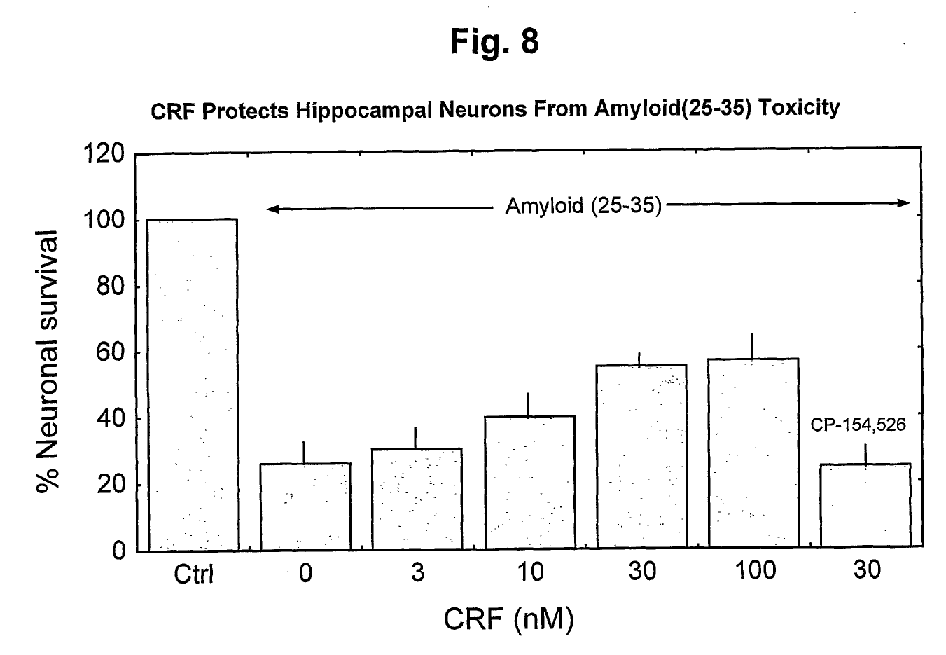

FIG. 8 is a bar graph illustrating percentage mean survival of hippocampal neurones when in the presence of the amyloid-β peptide (fragment 25-35) (Aβ) (10 μM), showing the effect of adding CRF at varying concentrations or both CRF and CP-154,526.

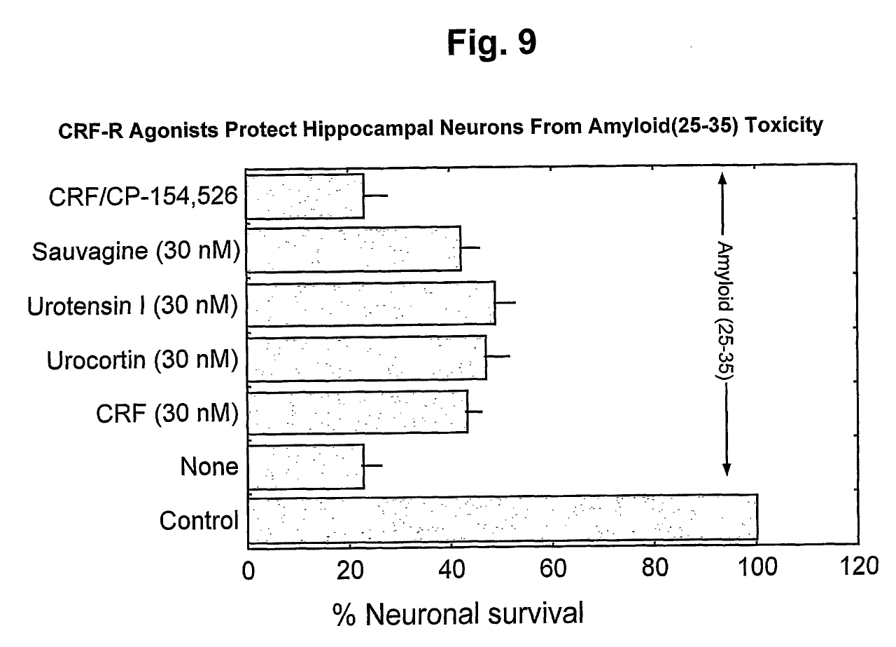

FIG. 9 is a bar graph illustrating percentage mean survival of hippocampal neurones when in the presence of the amyloid-β peptide (fragment 25-35) (Aβ) (10 μM), showing the effect of adding CRF receptor agonists at 30 nM (CRF, urocortin, urotensin 1, or sauvagine) or both CRF (30 nM) and CP-154,526 (1 μM).

### MATERIALS

The CRF receptor agonist peptides used in the tests were CRF, urotensin 1, urocortin and sauvagine. Specifically, the peptides used were all obtained from the Sigma catalogue, Sigma-Aldrich Company Ltd, Fancy Rd, Poole, Dorset, BH12 4QH, United Kingdom, and were:

Rat CRF (Sigma catalogue no. C-3042), with the sequence H-Ser-Gln-Glu-Pro-Pro-Ile-Ser-Leu-Asp-Leu-Thr-Phe-His-Leu-Leu-Arg-Glu-Val-Leu -Glu-Met-Thr-Lys-Ala-Asp-Gln-Leu-Ala-Gln-Gln-Ala-His-Asn-Asn-Arg-Lys-Leu-Leu-Asp-Ile-Ala-NH₂ (see also U.S. Pat. No. 4,489,163); CRF (human, rat) also available from Bachem, cat. no. H-2435 (S. Shibahara et al., EMBO J., 2, p. 775, 1983).

Urotensin 1 (teleost fish—Sigma cat. no. U-7253, or Bachem cat. no. H-5500), with the sequence H-Asn-Asp-Asp-Pro-Pro-Ile-Ser-Ile-Asp-Leu-Thr-Phe-His-Leu-Leu-Arg-Asn-Met-Ile-Glu-Met-Ala-Arg-Be-Glu-Asn-Glu-Arg-Glu-Gln-Ala -Gly-Leu-Asn-Arg-Lys-Tyr-Leu-Asp-Glu-Val-NH₂;

Urocortin (rat—Sigma Cat. no. U-663 1, lot no. 98H4954), with the sequence H-Asp-Asp-Pro-Pro-Leu-Ser-Ile-Asp-Leu-Thr -Phe-His-Leu-Leu-Arg-Thr-Leu-Leu-Glu-Leu-Ala-Arg-Thr-Gln-Ser-Gln-Arg-Glu-Arg-Ala-Glu-Gln-Asn-Arg-Ile-Ile-Phe-Asp-Ser-Val-NH₂ (see also WO 97/00063 and J. Vaughan et al., Nature, 378, 287-292, 1995); and

Sauvagine (frog—Sigma cat. no. S-3884, lot no. 97H10851), with the sequence pGlu-Gly-Pro-Pro-Ile-Ser-Ile-Asp-Leu-Ser-Leu-Glu-Leu-Leu-Arg-Lys-Met-Ile-Glu-Ile-Glu-Lys-Gln-Glu-Lys-Glu-Lys-Gln-Gln-Ala-Ala-Asn-Asn-Arg-Leu -Leu-Leu-Asp-Thr-Ile-NH₂ (see also P. C. Montecucchi and A. Henschen, Int. J. Peptide Protein Res., 18, 113, 1981; V. Espamer et al., Regulatory Peptides, vol. 2, (1981), pp 1-13; V. Erspamer and P. Melchiorri, Trends Pharmacol. Sci., 2, 391, 1980)

It should be noted that CRF, urotensin 1, urocortin and sauvagine derived from other sources (e.g. as indicated in the references mentioned in the introduction) can also be used.

The PI 3-kinase inhibitor LY 294002 is 2-(4-morpholinyl)-8-phenyl-4H-1-benzopyran-4-one and completely and specifically abolishes PI 3-kinase activity (IC₅₀=0.43 μg/ml; 1.40 μM) as described in C. J. Vlahos et al., J. Biol. Chem., 269, 5241-5248, 1994 (see especially Table 1, FIG. 1 and references 38 and 39 therein for preparation). In this case, LY 294002 was purchased from Calbiochem., Nottingham, United Kingdom, cat. no. 440202.

MTT is 3-(4,5-dimethylthiazol-2-yl)-2,5-diphenyltetrzolium bromide (T. Mosmann, J. Immunol. Methods 65, 55-63, 1983; M. Manthorpe et al., Dev. Brain Res. 25, 191-198, 1986; S. D. Skaper et al. in Methods in Neurosciences, Vol. 2 (Conn P. M., ed), pp.17-33. Academic Press, San Diego, 1990).

IBMX is 3-isobutyl-1-methylxanthine, obtainable from Calbiochem., cat. no. 410957. (cAMP is hydrolysed by phosphodiestersases, leading to cessation of cAMP-dependent effects. IBMX is a non-specific inhibitor of cAMP phosphodiesterases, and thus prevents or inhibits the breakdown of cAMP. Refs: Scamps, F., et al, Eur. J. Pharmacol (1993) 244:119-125. Turner, N C., et al, Br. J. Pharmacol. (1993) 108:876-882.)

The structure, methods of synthesis and biological profile of CP154,526 are described in WO 94/13676 (Pfizer), D. W. Schultz et al., Proc. Natl. Acad. Sci., USA, 93, 10477, 1996 and Y. L. Chen et al., J. Med. Chem., 40, 1749-1754, 1997; and is reviewed in McCarthy J R et al., Current Pharmaceutical Design (1999) 5:298-315 and in P. J. Gilligan et al, J. Med. Chem., 43(9), 1641-1660, 2000, see pages 1650-1. CP154,526 is a highly selective CRF receptor-1 antagonist.

Rp-cAMPS is described in Gjertsen B T et al, J. Biol. Chem (1995) 270:20599-20604 and is commercially available from Calbiochem, Nottingham, catalogue number 116816. It is an inhibitor and isomer of cAMP.

Experimental Protocols

Cerebellar granule neurones were used in the examples to test neuroprotection by CRF receptor agonists. Cerebellar granule neurones (CGNs) undergo apoptosis during the first few weeks of postnatal life, and in culture require mildly depolarising concentrations of KCl (25 mM) for their survival and maturation (S. R. D'Mello et al., Proc. Natl. Acad. Sci. USA 90, 10989-10993, 1993). The growth signal provided by KCl depends upon activation of the phosphatidylinositol 3-kinase (PI 3-kinase) pathway, and pharmacological inhibition of PI 3-kinase in differentiated granule neurones leads to apoptotic death in the CGNs (T. M. Miller et al., J. Biol. Chem., 272, 9847-9853, 1997). Thus, cultured CGNs represent a suitable model to study mechanisms of programmed cell death, and to identify putative neuroprotective signalling pathways.

Neuroprotection Assays

Assay 1: Cerebellar granule neurones prepared from postnatal day 8 Sprague Dawley rat pups were used for experiments on death induced by inhibition of PI 3-kinase. General culture methods are described in S. D. Skaper et al. in Methods in Neurosciences, Vol. 2 (Conn P. M., ed), pp.17-33. Academic Press, San Diego, 1990. At 8-9 days in vitro (DIV), granule neurone cultures were shifted to phenol red-free Dulbecco's modified Eagle's medium lacking serum, and containing 0.05% bovine serum albumin and 25 mM KCl. Death was induced by addition of the PI 3-kinase inhibitor LY 294002 (75 μM). CRF or CRF agonist peptides (urotensin 1, urocortin, sauvagine) were added at different concentrations (from 0.3 nM to 300 nM or 1000 nM) together with LY 294002. Forty-eight hours later neuronal survival was quantified by a colorimetric reaction MTT. Absolute MTT values obtained were normalised for small differences in interexperiment plating densities by scaling to the mean of sham-treated sister cultures (defined as 100%).

Results are shown in Table 1 below and in graphical form in FIG. 1, which show the percentage mean neuronal survival (and standard deviation), when in the presence of the relevant CRF receptor agonists and LY 294002, as a function of agonist concentration. It can be seen that CRF receptor agonists provided neuroprotection in a concentration-dependent manner, with CRF being the most potent (EC₅₀=about 10 nM). Urotensin 1, urocortin and sauvagine were also neuroprotective albeit with lower potency (<1 μn.

The relative effectivness of the CRF receptor agonists at 10 nM concentration can also be seen in the bar chart in FIG. 4, as discussed later.

TABLE 1
neuroprotection results corresponding to FIG. 1
CRF

| | | | | | | | |
|---|---|---|---|---|---|---|---|
| Mean | 82 | 96.11 | 95 | 96 | 58.11 | 49 | 55 |
| survival % | | | | | | | |
| Standard | 1.73 | 17.11 | 22.68 | 16.17 | 17.94 | 3.29 | 7.75 |
| deviation | | | | | | | |
| Compound | CRF | CRF | CRF | CRF | CRF | CRF | CRF |
| Concentration | (300 nM) | (100 nM) | (30 nM) | (10 nM) | (3 nM) | (1 nM) | (0.3 nM) |

UROTENSIN 1

| | | | | | | | | |
|---|---|---|---|---|---|---|---|---|
| Mean | 79.17 | 73.83 | 71.17 | 71.67 | 74.44 | 68 | 52.89 | 42 |
| survival % | | | | | | | | |
| Standard | 1.72 | 6.27 | 8.84 | 7.20 | 4.85 | 9.79 | 6.62 | 2 |
| deviation | | | | | | | | |
| Com- | Uro- | Uro- | Uro- | Uro- | Uro- | Uro- | Uro- | Uro- |
| pound | tensin | tensin | tensin | tensin | tensin | tensin | tensin | tensin |
| Concen- | (1000 nM) | (300 nM) | (100 nM) | (30 nM) | (10 nM) | (3 nM) | (1 nM) | (0.3 nM) |
| tration | | | | | | | | |

UROCORTIN

| | | | | | | | |
|---|---|---|---|---|---|---|---|
| Mean | 84 | 75 | 80.67 | 77.33 | 69.33 | 61.33 | 50.67 |
| survival % | | | | | | | |
| Standard | 1 | 14 | 10.21 | 4.93 | 2.58 | 3.01 | 4.62 |
| deviation | | | | | | | |
| Compound | Uro- | Uro- | Uro- | Uro- | Uro- | Uro- | Uro- |
| Concentration | cortin | cortin | cortin | cortin | cortin | cortin | cortin |
| | (300 nM) | (100 nM) | (30 nM) | (10 nM) | (3 nM) | (1 nM) | (0.3 nM) |

SAUVAGINE

| | | | | | | | |
|---|---|---|---|---|---|---|---|
| Mean | 78.33 | 77.33 | 78.67 | 80.67 | 76.33 | 66.5 | 49.33 |
| survival % | | | | | | | |
| Standard | 2.31 | 2.31 | 2.89 | 3.50 | 6.09 | 6.72 | 2.89 |
| deviation | | | | | | | |
| Compound | Sauvagine | Sauvagine | Sauvagine | Sauvagine | Sauvagine | Sauvagine | Sauvagine |
| Concen- | (300 nM) | (100 nM) | (30 nM) | (10 nM) | (3 nM) | (1 nM) | (0.3 nM) |
| tration | | | | | | | |

Assay 2: This assay measures the effects of CP154,526, a selective CRF receptor-1 antagonist, on the protective effects of a putative CRF receptor agonist against neurotoxicity induced by LY294002 in primary cerebellar granule neurones. The assay is identical to Assay 1 except that after shifting the granule neurone cultures to the medium, the cultures were incubated with CP154,526 at concentrations varying from 3 nM to 1000 nM for 30 minutes prior to simultaneous addition of the putative CRF receptor agonist (10 nM) and LY 294002 (75 μM). 48 hours later neuronal survival was measured as in Assay 1.

FIG. 2A shows the results for the agonist CRF (10 nM), measured as neuronal survival as a percentage of controls where no compounds were added (values shown are the mean±standard deviation). The columns from left to right represent: (1) control: cells cultured with CRF (10 nM) and LY 294002 (75 μM); (2 to 7) cells cultured with CRF (10 nM), LY 294002 (75 μM) and CP 154,526 at concentrations of 3 nM, 10 nM, 30 nM, 100 nM, 300 nM and 1000 nM respectively; and (8) cells cultured without agonist (CRF) but with LY 294002 (75 μM).

It can be seen from FIG. 2A that CP 154,526 appears to remove almost completely the neuroprotective effect of 10 nM CRF at concentrations as low as 300 nM. This suggests that the neuroprotective effects of CRF itself are mediated almost exclusively through CRF receptor-1.

Further, similar patterns to that shown with CRF as agonist are seen for the corresponding tests shown in FIGS. 2B, 2C and 2D where CRF is replaced by urocortin, urotensin I, and sauvagine respectively (the compound(s) present in the 8 columns in each of these three Figures are the compound(s) present in the corresponding 8 columns in FIG. 2A with CRF being replaced by the appropriate agonist as necessary). In each of FIGS. 2B-D, a 1000 μM concentration of CP 154,526 appears to remove most or all of the neuroprotective effect of 10 nM CRF receptor agonist, and partial reductions in neuroprotection appear to be seen at 300 nM CP 154,526.

These results seem to suggest that the CRF receptor-1 antagonist CP 154,526 inhibits the ability of CRF agonists (in general) to protect cultured cerebellar granule neurons from death induced by the PI 3-kinase inhibitor LY 294002, and that the neuroprotective effects of CRF receptor agonists in general are mediated mainly through CRF receptor-1.

Cyclic AMP Measurements

Assay 3: The production of cAMP by the CRF receptor agonist peptides CRF, urotensin 1, urocortin and sauvagine was measured as follows. This assay can be used analogously for any putative CRF receptor agonists including non-peptide agonists.

Intracellular cyclic AMP was measured with a cAMP 2-site enzyme immunoassay system (trade mark BIOTRAK, commercially available from Amersham Pharmacia Biotech, PO Box 164, Rainham, Essex RM13 8JZ, United Kingdom, Amersham catalogue number RPN225), based upon the use of a sensitive and highly specific capture antibody for cyclic AMP:

Cerebellar granule neurones were seeded in polylysine-coated 96-well plates, 3.5×10⁵ cells per 48-well, in Basal medium Eagle's containing 10% fetal calf serum, 25 mM KCl, and antibiotics. At 8-9 days in vitro, granule neurone cultures were shifted to serum-free plating medium (0.4 ml) with 0.5 mM IBMX (to inhibit the breakdown of cyclic AMP) for 15 min (37° C.). Wells then received 0.1 ml of test peptide (5× final concentration). Generally 1 μM is the preferable final concentration for a putative CRF agonist under test, though different (e.g. lower) concentrations can be used as shown for the peptide agonists in FIG. 3. Incubation was continued for 15 min. Test medium was removed and the plate placed on dry ice, and stored at −140° C. Cyclic AMP analysis was performed the following day.

Cells were then lysed with the reagent provided with the BIOTRAK kit. Intracellular content of cyclic AMP in the lysates was assayed following the manufacturer's instructions. A standard curve was constructed using the cyclic AMP calibration provided, with the quantity of bound cyclic AMP being inversely proportional to the amount of second (chromogenic) antibody bound to the reaction wells. The colour product was read with an ELISA plate reader, and was a function of the amount of cyclic AMP in the Renown sample.

FIG. 3 illustrates the cAMP synthesis induced by CRF, urotensin 1, urocortin or sauvagine measured in Assay 3 in absolute values of cAMP per cell number, in primary cerebellar granule neurones, as a function of agonist concentration varying from 0 to 30 nM. From this dose-response curve, it can be seen that cAMP production increased with agonist concentration.

Assay 4: The test compounds stimulating cAMP production by more than a threshold multiplier, e.g. 5 times, compared to controls in Assay 3 above can optionally be selected for a second screen (Assay 4), to test whether the cAMP production caused by the a test compound is primarily caused by stimulation of CRF receptor-1. In this assay, cAMP production by the test compound is measured in cerebellar granule neurones in the presence of the selective CRF-R1 antagonist CP154,526—if cAMP production mediated by the putative CRF receptor agonist is suppressed by the presence of CP154,526 then this indicates CRF receptor-1 agonist activity.

The assay is identical to Assay 3 except that the cultures were incubated with CP154,526 at a supramaximum concentration (100 μM, or generally in ca. 100-fold excess over the CRF agonist) for 15 minutes prior to addition of the putative CRF receptor agonist (preferably at 1 μM final concentration, though other concentrations may be used). Incubation was then continued for 15 minutes further, and processing and cAMP analysis was conducted as in Assay 3.

As CP154,526 binds CRF receptor-1 competitively, the final concentration of CP154,526 in Assay 4 should be much greater (preferably at least 100 times greater) than the agonist concentration to ensure that most of the CRF-1 receptors are bound by CP154,526 and that there is very little competitive binding of the CRF-1 receptors by the putative agonist.

Involvement of Cyclic AMP in CRF Receptor Agonist Neuroprotection—Discussion

From the results using Assay 3 shown in FIGS. 3, it can be noted that the neuroprotection mediated by CRF receptor agonists illustrated in FIG. 1 appears to be associated with production of cyclic AMP. (cAMP is believed to protect cerebellar granule neurones from apoptotic death as discussed above and in S. R. D'Mello et al., Proc. Natl. Acad. Sci. USA 90, 10989-10993, 1993).

Further evidence has also been found that the neuroprotective efficacy of CRF and its analogues does rely to an extent on this rise in cAMP. The application of Rp-cAMPS (an inhibitor and isomer of cAMP) was found to reduce the extent of (i.e. partially antagonised) neuronal rescue provided by the CRF receptor agonists by about 20%, as illustrated in FIG. 4.

FIG. 4 illustrates percentage mean survival of primary cerebellar granule neurones, when in the presence of the PI 3-kinase inhibitor LY 294002 (75 μM) and also a CRF receptor agonist at 10 nM (CRF, urocortin, urotensin 1, or sauvagine), in the absence or presence of the cAMP inhibitor Rp-cAMP (100 μM). The columns from left to right represent: (1) control without LY 294002 or agonist; (2) control without agonist but with LY 294002; (columns 3, 5, 7, 9) with LY 294002 and either CRF, urocortin, urotensin 1, or sauvagine respectively; (columns 4, 6, 8, 10) as columns 3, 5, 7 and 9 respectively but additionally also with Rp-cAMP. The approx. 20% reduction in neuroprotection when in the presence of Rp-cAMP is clear.

Overall, the data presented in FIGS. 1 to 4 and Table 1 herein suggest that CRF receptor activation, and subsequent engagement of cAMP-dependent signalling pathways, may provide neurotrophic support in vivo for cerebellar granule neurones and neuronal cells in general. However, the failure of Rp-cAMP to completely suppress the protective activity of CRF receptor agonists suggests that another (as yet unknown) messenger system other than cAMP is also mediating the protective effects of CRF receptor stimulation, perhaps via interaction with GSK-3 (see below) and/or another part of the PI 3-kinase signalling pathway.

Evidence that CRF Receptor Agonists Interact with the PI 3-Kinase Pathway at Least in Part by Phosphorylation and thus Inhibition of the Pro-apoptotic Protein GSK-3

It was thought that CRF receptor agonists might interact with the PI 3-kinase signalling pathway and exert their neuroprotective effect by phosphorylating and inhibiting the protein GSK-3β, which is pro-apoptotic (M. Pap and G. M. Cooper, J. Biol. Chem., 273(32), 19929-19932, 1998; and M. Hetman et al. J. Neurosci., Apr. 1, 2000, 20(7), 2567-2574; discussed hereinabove). In order to confirm this, the level of GSK-3β phosphorylated at serine-9 (e.g. see D. A. E. Cross, Nature, 378, 785-789, 1995) in control cultures of cerebellar granule neurones was measured using Western blots, and compared to the level of phospho-GSK-3β in the presence of CRF as a model CRF receptor agonist and/or in the presence of the PI 3-kinase inhibitor LY 294002. In order to determine whether elevation of cAMP was involved in any effect, the level of phospho-GSK-3β in cultures in the presence of the known cAMP-elevating agent forskolin (FSK, obtainable e.g. from Calbiochem.) with or without LY 294002 present was also measured. The forskolin acts as a benchmark for cAMP-related effects.

The method used was as follows. Cerebellar granule neurone cells were cultured for 8 days in: Basal Media Eagle (BME), plus 10% foetal calf serum, 25 mM KCl and the antibiotic gentamicin (“complete media”). Thereafter, the medium was aspirated and replaced with 25 mM KCl-containing culture medium (control medium “CN”: BME plus penicillin/streptomycin, but without the serum) containing the appropriate compound or compounds (as shown in FIG. 5) for 2 hours in incubators. Thereafter, solutions were aspirated and cell lysis buffer was added (1% Triton X-100 0.5% SDS, 0.75% deoxycholate, 10 mM Tris Base pH 7.0, 75 mM NaCl, 10 mM EDTA, 0.5 mM PMSF, 2 mM sodium orthovanadate, 10 μg/ml aprotinin, 1.25 mM NaF, 1 mM sodium pyrophosphate.). The samples were spun at 13,000 RPM at 4° C., 150 μl were resuspended in 30 μl Laemmli buffer and 15 μl loaded per lane in the Western blot. Proteins were size fractionated by sodiumdodecylsulfate-polyacrylamide gel electrophoresis (SDS-PAGE) and transferred to polyvinyldifluoride (PVDF) membranes. Membranes were blocked in blocking solution (5% milk in TBS/T [20 mM Tris base pH 7.6, 150 mM NaCl, 0.1% Tween-20]).

Phosphorylation of GSK-3β was detected using an antibody to serine-9-phosphorylated GSK-3β (anti-phospho-GSK-3β (Ser9)) available as catalogue no. 9336 from Cell Signalling Technology, 166B Cummings Center, Beverly, Mass.01915, USA or from the sister-company New England Biolabs (UK) Ltd, 73 Knowl Place, Wilbury Way, Hitchin, Hertfordshire SG4 0TY, United Kingdom. Total GSK-3β was detected using an antibody to total GSK-3β (Transduction Laboratories, 133 Venture Court, Lexington KY 40511-2624, USA) to demonstrate loading levels. Anti-phospho-GSK-3β antibodies were used at a concentration of 1:1000 (volume ratio of antibody to blocking solution) of stock diluted in block solution. The antibody to total GSK-3beta was used at 1:2500 concentration in the same diluent. Secondary antibodies were used as follows:

For anti phospho-GSK3β, HRP-conjugated anti-rabbit IgG (H+L) (available from Promega Corp., 2800 Woods Hollow Road, Madison, Wis. 53711-5399, USA, catalogue no. V7951) was used at concentrations of 1:7500 in blocking solution. HRP =horseradish peroxidase.

For total GSK-3β, HRP-conjugated anti-mouse IgG (H+L) (Promega catalogue no. W4021; alternatively available from Pierce) was used at 1:2500 concentration in blocking solution.

Detection was by enhanced chemiluminescence (Amersham Pharmacia Biotech, PO Box 164, Rainham, Essex RM13 8JZ, United Kingdom).

The results are shown in FIG. 5, at the top of which is a Western blot electrophoresis gel showing levels of serine-9-phosphorylated GSK-3β (phospho-GSK-3β) and total GSK-3β in cerebellar granule neurones cells in the presence of (from right to left): complete medium; control serum-free medium (CN); CRF (10 nM); LY 294002 (75 μM); CRF (10 nM)+LY 294002 (75 μM) added together; forskolin (FSK) (30 μM); and LY 294002 (75 μM)+FSK (30 μM) added together. The fold induction of phospho-GSK-3β compared to control CN=1 for each lane is shown in a bar graph aligned lane-by-lane below. It can be seen from FIG. 5 that CRF alone gives an increase in basal phospho-GSK-3β and the PI 3-kinase inhibitor LY 294002 a decrease compared to control. In the presence of both CRF and LY 294002, phospho-GSK-3β levels are similar to and slightly higher than those for CRF alone. Phospho-GSK-3β levels with FSK are raised higher than for CRF, and again are not affected by LY 294002.

These results show (as expected) that PI 3-kinase inhibition leads to decreased GSK-3β phosphorylation which leads to increased activiation of GSK-3β. The CRF and CRF+LY data are evidence suggesting that CRF receptor agonists mediate the serine-9 phosphorylation of GSK-3β, to an extent substantially independent of the presence or absence of PI 3-kinase inhibitor. This suggests that CRF receptor agonists in general at least in part rescue the PI 3-kinase cell-saving pathway and exert their neuroprotective effect by GSK-3β phosphorylation and inhibition, perhaps without greatly influencing Akt or PI3K. The forskolin (FSK) results being qualitatively similar suggest that at least in part the CRF agonists are acting on GSK-3β by raising cAMP levels, like FSK.

It is thus postulated that CRF receptor agonists work in part by raising cAMP, which is known to activate protein kinase A, the protein kinase A in turn phosphorylating and inhibiting GSK-3β (analogously to Akt) thereby reducing/mitigating apoptosis. However, the amount of GSK-3β phosphorylation by CRF is only modest compared to FSK (a strongly cAMP-elevating agent). Taken together with the results in FIG. 4 described above, in which a cAMP inhibitor only partially suppressed the neuroprotective activity of CRF agonists, it seems likely that one or more messenger systems other than cAMP elevation and/or GSK-3β phosphorylation are also involved in the neuroprotective effect of CRF receptor agonists. For example, it is possible that CRF agonists could also be neuroprotective by causing the phosphorylation and/or inhibition of other pro-apoptotic proteins such as BAD (T. F. Gajewski et al., Cell, 87, 589, 1996; S. R. Datta et al., Cell, 91, 231-241, 1997) which are downstream of Akt and/or PI3K. The interaction of CRF agonists with BAD is currently being investigated using similar (anti-phospho-BAD) antibody and Western blotting techniques analogous to the anti-phospho-GSK-3β experiment shown in FIG. 5 and described above. Finally, preliminary results with anti-phospho-Akt antibodies and Western blot analysis, again analogous to the anti-phospho-GSK-3β experiment shown in FIG. 5, suggest that interaction of the CRF receptor agonists with Akt is not very likely to be occurring (little recovery of Akt activity was observed with CRY).

Delayed Addition of CRF Receptor Agonists

It has also been found that CRF can be added some time after the PI 3-kinase inhibitor LY 294002 has been added and still achieve its neuroprotective effect, i.e. that delayed CRF addition is sufficient to protect cerebellar granule neurons from injury by LY 294002.

FIGS. 6A and 6B show neuronal survival results, using a variation of Assay 1 in which CRF (10 nM) was added at varying times (shown in hours) following LY 294002 (75 μM) addition. Therefore, granule neurons were cultured with the PI 3-kinase inhibitor LY 294002 (75 μM) in the presence of 10 nM CRF, added together with LY 294002 or at different times (shown in hours) following LY 294002 addition. FIG. 6A is the result from one experiment only. FIG. 6B is the combined result from two experiments, and shows the effect of the delayed addition of CRF at different times over a longer (46 hour vs. 32 hour) timecourse. It appears from FIG. 6A that the neuroprotective effect is sustained when adding CRF at about 1-4 hrs after LY 294002 addition. From FIG. 6B, it appears that the neuroprotective effect is sustained when adding CRF up to 8 hours after LY 294002 addition. Only a small neuroprotective effect is seen when adding CRF at 10, 24 and 32 hours after LY 294002 addition, and no neuroprotection is seen when adding CRF at 46 hours after LY 294002 addition (neuronal survival returns to the level seen with LY 294002 alone—see the horizontal line in FIG. 6B).

This suggests that there may be a reasonably large window for therapeutic intervention between an acute occurrence in a patient (e.g. traumatic/mechanical brain injury, stroke, etc.) which potentially leads to neuronal damage and the later administration of a CRF receptor agonist to the patient (e.g. in hospital).

Use of the Assays for Determining CRF Receptor Agonist Activity

The cAMP Assay 3 can be used as a general method of determining CRF receptor agonist activity, as described hereinbefore. A screen for selecting potential lead compounds having CRF receptor agonist activity can be constructed by measuring and selecting those compounds which stimulate cAMP production by (for example) more than 5 times compared to controls.

Assay 4 can be used as a second screen, to screen lead compounds already testing positive in Assay 3 for CRF receptor-1 agonist activity, and/or to confirm that the cAMP production observed for that compound in Assay 3 is mediated via stimulation of CRF receptor-1. If cAMP production mediated by the test CRF receptor agonist in Assay 3 is suppressed by the presence of CP154,526 in Assay 4, then this indicates CRF receptor-1 agonist activity.

An optional third screen confirming CRF receptor-1 agonist activity would be to run the test compound in Assay 2 to compare the neuroprotection conferred by the CRF receptor agonist in the presence of LY 294002 with that conferred when also in the presence of CP154,526—a decrease in neuroprotection here confirms that neuroprotection by the test compound is mediated via stimulation of CRF-R1.

Alternatively, instead of Assays 3 and/or 4, to measure CRF receptor-1 agonist activity, cells (e.g. CHO cells) stably transfected with CRF-R1 (e.g. see R. Chen et al., Proc. Natl. Acad. Sci. USA, 90, 8967-8971, 1993; J. Vaughan et al., Nature, 378, 287-292, 1995, Table 1 and references cited in these two articles) can be subjected to the putative CRF receptor ligands and intracellular cAMP production can be measured (similar to in Assay 3) as a measure of CRF-R1 stimulation. To measure selectivity, this would be followed by cross-screens with cells stably transfected with CRF-R2α and/or R2β (e.g. see WO 95/34651, pages 43 to 48), again using cAMP as a measure of stimulation of those receptors. To confirm that the cAMP production is primarily caused by stimulation of CRF receptor-1, cAMP production by the test compound could be measured using a modification of both Assays 3 and 4 above.

Similarly, in a general screen of agonist activity versus CRF receptors in general (as opposed to CRF-R1), compounds testing positive in the cAMP production Assay 3 could be optionally subjected to a second assay similar to Assay 4 but wherein CP 154,526 is replaced by a non-selective CRF-receptor antagonist (i.e. which antagonises all CRF receptors or at least type-1, 2α and 2β receptors). If cAMP production mediated by the putative CRF receptor agonist under test is suppressed by the presence of the CRF receptor antagonist then this indicates a general CRF receptor agonist activity. Suitable CRF receptor antagonists for this purpose include: astressin [available from Sigma (cat. no. A4933), see also J. Gulyas et al., Proc. Natl. Acad. Sci. USA, 92, p10575, 1995 and refs. cited therein]; compound 49 mentioned on page 1652 of P. J. Gilligan et al, J. Med. Chem., 43(9), 1641-1660, 2000 and described in U.S. Pat. No. 5,861,398 and D. R. Luthin et al., Bioorg. Med. Chem. Lett., 9, 765-770, 1999 (a combined CRF-R1 and CRF-R2 antagonist); and possibly the pyrimidine derivatives disclosed in EP 0976745 A1 (Taisho Pharmaceuticals).

Reference is also made to Assays 5 and 6 hereinafter which give guidance as to whether, in more specific in vitro or in vivo situations involving or mimicing cerebral ischaemia or amyloid-β peptide/Alzheimer's disease, CRF receptor agonists confer neuroprotection by stimulating CRF receptor-1.

Effects of Administration of Intracerebroventricular (icv) Urotensin I Following Distal Middle Cerebral Artery Occlusion (MCAO) in Spontaneous Hypertensive Rats (SHR)—FIG. 7

The aim of this study was to investigate the effects of icv urotensin I in a distal occlusion model (a type of experimentally induced cerebral ischaemia or stroke) in spontaneous hypertensive rats (SHR), and to confirm that CRF receptor agonists are neuroprotective in animal models of stroke/cerebral ischaemia in vivo. The animal protocols are as follows. The results are shown in FIG. 7.

Surgical Focal Ischemia Preparation

Focal ischemia experiments were performed on male spontaneously hypertensive rats (SHR; Taconic Farms, Germantown, N.Y., US) weight range 280-340 g. Body temperature was maintained at 37° C. during all surgical procedures and during recovery from anesthesia (i.e., until normal locomotor activity returned). Animals were anesthetized with pentobarbital (65 mg/kg, i.p.) and underwent permanent, right middle cerebral artery occlusion (MCAO) for 24 h as described previously (F. C. Barone et al., Neuroscience & Biobehavioral Reviews, 16 (1992) 219-33, and F. C. Barone et al., Stroke, 29 (1998) 1937-50). Body temperature was monitored throughout the surgical procedure by a rectal thermometer, and the animals maintained normothermic (37±0.5° C.) via a heating blanket controlled by the thermometer. A needle temperature probe was also inserted into the left temporalis muscle to give an indirect measurement of brain temperature. Actual core and temporalis temperature values were recorded at the time of MCA occlusion.

ICV Cannulae

Rats were implanted with icv cannulae prior to surgery.

Post-Occlusion Recovery

The rats were allowed to recover from surgery on a heating pad while containing to be under the influence of the pentobarbital anesthesia Once animals were able to right themselves and begin spontaneous movement, they were placed in cages on the heating pads and monitored for any distress until fully recovered from anesthesia.

Neurological Assessment

After 24 h of permanent MCAO each rat then was evaluated for neurological deficits using two graded scoring systems as previously described (Barone et al., 1992; 1998—see above for refs). Briefly, forelimb scores were zero (no observable deficit), one (any contralateral forelimb flexion when suspended by the tail) and two (reduced resistance to lateral push towards the paretic, contralateral side. A hindlimb placement test consisted of pulling the contralateral hindlimb away form the rat over the edge of a table. A normal response (zero score) is an immediate repositioning of the limb back onto the table and an abnormal/deficit response (one score) is no limb placement/movement. The total score (i.e., the sum) of both tests was utilized as a global neurological deficit score for each rat.

Neuropathology and Quantification of Ischemic Damage

Rats were then euthanized (killed) by an overdose of sodium pentobarbital (200 mg/kg, i.p.). The brains were immediately removed and 2-mm coronal sections were cut from the entire forebrain area (i.e. from the olfactory bulbs to the cortical- cerebellar junction), using a brain slicer (Zivic-Miller Laboratories). The coronal sections were immediately stained in a solution of 1% triphenyltetrazolium chloride as described previously (Barone et al., 1992; 1998—see above). Sections were transferred to 10% formalin (in 0.1% sodium phosphate buffer) for at least 24 h and then photographed and analyzed also as described previously (Barone et al., 1992; 1998). Briefly, brain injury was quantified using an Optimus image analysis system (DataCell) and the degree of brain damage will be corrected for the contribution made by brain edema/swelling as described previously (Barone et al., 1998). Hemispheric swelling and infarct size (infarct=dead brain tissue) was expressed as the percent infarcted tissue in reference to the contralateral hemisphere, and infarct volume (mm³) was calculated from the infarct areas measured on from the sequential forebrain sections.

Dosing

Vehicle or drug was administered 15 min and 2 hours post MCAO:

1. Vehicle=isotonic saline

2. Drug=urotensin I 10 μg (10 micrograms/rat) icv (injection into cerebral ventricles)

Results are shown in FIG. 7, and show that whereas the size of the infarct (dead brain tissue) for vehicle-treated rats was about 100 mm³, infarct size for the urotensin I-treated rats was less, at about 70-80 mm³. This appears to be a significant reduction in infarct size. Also, the numerical scores for neurological deficits were higher in vehicle-treated rats (11) than for urotensin I-treated rats (9). The results overall appear to suggest that urotensin I, and CRF receptor agonists in general, are neuroprotective in in vivo animal models of stroke/cerebral ischaemia.

Assay 5. The MCAO test given above and in FIG. 7 can also be modified to confirm or test that any CRF agonist under test, e.g. one of the 4 exemplified peptide agonists such as urotensin I, is working by stimulation of CRF receptor, in particular by stimulating CRF receptor-1. The test conditions are analogous, but instead of adding the CRF agonist alone, one would add the test agonist as well as the selective CRF receptor-1 antagonist CP154,526 described above. Preferably, one would administer the CP154,526 by a suitable route e.g. by icv (injection into cerebral ventricles), say 5-15 mins before MCAO and then administer the agonist under test 15 Minutes and 2 hours post-MCAO (as for urotensin I above). Alternatively the timings can be altered but preferably the CP154,526 is administered at least 15-30 minutes before the putative CRF receptor agonist is given to allow the CRF-1 receptors to be blocked. If the CP 154,526 abolishes any neuroprotection conferred by the putative CRF receptor agonist, then this test compound should be working via stimulation of CRF receptor-1. For a general test of treating ischaemia by stimulation of any CRF receptor, one could use astressin or a similar non-selective CRF receptor antagonist in place of CP154,526.

CRF Protects Neurones from β-amyloid(25-35) Toxicity—FIGS. 8 and 9

The β-amyloid protein (amyloid β-protein, Aβ), usually containing 1-42 or 1-40 amino acids, is neurotoxic. Aβ is a major component of senile plagues in Alzheimer's disease (AD) patients, and a long-standing hypothesis is that aberrant accumulation of Aβ occurs in AD brain and is associated with formation of neurofibrillatory tangles and neuronal death (J A Hardy et al., Science, 256, 184-185, 1992; D J Selkoe, Annu Rev Neurosci., 12, 463-490, 1989; M G Spillantini et al., Proc Natl Acad Sci USA, 87, 3947-3951 and 3952-3956, 1990). The neurotoxic sequence of Aβ is the 25-35 amino acid stretch. Several studies have linked the PI 3-kinase pathway, in particular GSK-3, with Alzheimer's disease—see discussion above and C. C. Weihl et al., J. Neurosci., 19, 5360-5369, 1999; A. Takashima et al., Neuroscience Letters, 203, 33-66, 1996; A. Takashima et al., Proc. Natl. Acad. Sci. USA, 90, 7789-7793, 1993, see p. 7789 and conclusion on p. 7792; M. Hong and V. M. -Y. Lee, J. Biol. Chem., 272(31), 19547-19553, 1997 and references 14-16, 21 and 22 cited therein; WO 00/21927; WO 00/38675; WO 01/09106; and WO 98/16528.

In order to test whether CRF receptor agonists protect neurones from death caused by Aβ, the following experiment was conducted (FIGS. 8 and 9). Hippocampal neurons from embryonic day 18 rat were cultured in serum-free medium (neurobasal/B27) for 9 days. The culture methods are described on page 48 of S. D. Skaper et al., J. Neurochem., 2001, 76, 47-55. Cells were then treated with amyloid-β peptide (fragment 25-35) (Sigma, cat. no. A-4559, sequence Gly-Ser-Asn-Lys-Gly-Ala-Ile-Ile-Gly-Leu-Met) at 10 μM plus the indicated CRF receptor agonist at the indicated concentration, in the same culture medium. In some cases, the CRF-R1 antagonist CP-154,526 (1 μM) was added together with 30 nM CRF/CRF agonist. Cell survival was assessed after 3 days, by fixing the cultures and counting microscopically viable neurons. Values are expressed relative to numbers of surviving neurons in untreated cultures (=100).

FIG. 8 shows the results when using the above method, being a bar graph illustrating percentage mean survival of hippocampal neurones when in the presence of the amyloid-β peptide (fragment 25-35) (Aβ) (10 μM), showing the effect of adding CRF at varying concentrations or both CRF and CP-154,526. The lanes are as follows from left to right: (i) control, (ii) Aβ alone, (iii-vi) Aβ combined with CRF at 3, 10, 30 and 100 nM concentrations, (vii) Aβ with CRF (30 nM) and CP-154,526 (1 μM). Lane (ii) shows the neurotoxicity of Aβ alone; lanes (iii-vi) show that CRF protects the cells partially from Aβ toxicity and in a concentration-dependent manner, and lane (vii) shows that CRF's neuroprotective effect appears to be caused by stimulation of CRF receptor-1 (CP-154,526 cancels all of CRF's neuroprotection). The partial (ca. 40-50%) neuroprotective effect of CRF may be explained by Aβ exerting its neurotoxicity by routes other than suppression of the PI 3-kinase pathway, e.g. via oxidative stress.

Similar results to those shown in FIG. 8 were obtained when using 20 μM Aβ (results not shown). It is noted that after 1 day of 10-20 μM Aβ exposure, there was no visible neuronal death, but cell death was visible after 2-3 days. Finally, the reverse β-amyloid sequence (35-25) at 20 μM was found not to be neurotoxic (results not shown), in accordance with published studies. Therefore, the caused cell death is not just general to the β-amyloid peptide, it is sequence-specific.

FIG. 9 is a bar graph illustrating percentage mean survival of hippocampal neurones when in the presence of the amyloid-β peptide (fragment 25-35) (Aβ) (10 βM), showing the effect of adding CRF receptor agonists at 30 nM (CRF, urocortin, urotensin 1, or sauvagine) or both CRF (30 nM) and CP-154,526 (1 μM). The lanes are as follows from bottom to top: (1) control, (2) “none”=Aβ alone, (3-6) Aβ combined with CRF, urocortin, urotensin 1, or sauvagine respectively at 30 nM concentrations, (7) Aβ with CRF (30 nM) and CP-154,526 (1 μM). This graph shows that the three exemplifed agonist peptides other than CRF have a similar neuroprotective effect to CRF.

The results show that CRF receptor agonists protect neurones from death caused by amyloid-β peptide, and lend further support to their potential as inhibitors of neuronal cell death in the treatment or prophylaxis of Alzheimer's disease.

Assay 6. Note that the above-described method leading to the results in FIGS. 8 and 9, in particular the comparisons of neuroprotection achieved with agonists alone and with agonist+CP-154,526, can be modified and used as appropriate by the skilled man to give evidence that a putative CRF agonist is operating by stimulating CRF-R1 (a) when combating Aβ toxicity and/or (b) during the treatment/prophylaxis of Alzheimer's disease.

1. The use of a CRF receptor agonist, or a pharmaceutically acceptable salt, complex or prodrug thereof, for the manufacture of a medicament for the prevention or inhibition of neuronal cell death in a mammal suffering from or susceptible to chronic neurodegenerative disease, traumatic (mechanical) neuronal injury, epilepsy-associated neuronal loss, paralysis, or spinal chord injury.

2. The use as claimed in claim 1 wherein the mammal is human and is suffering from or susceptible to Alzheimer's disease, Parkinson's disease or Huntington's disease.

3. The use as claimed in claim 1 wherein the mammal is suffering from or susceptible to Alzheimer's disease.

4. The use of a CRF receptor agonist, or a pharmaceutically acceptable salt, complex or prodrug thereof, for the manufacture of a medicament for the repair or regeneration of neuronal cells in a mammal.

5. The use as claimed in any one of claims 1 to 4, wherein the CRF receptor agonist is a CRF receptor-1 agonist.

6. The use as claimed in claim 5, wherein the neuronal cell death is prevented or inhibited, or the neuronal cells are repaired or regenerated, by stimulating CRF receptor-1.

7. The use of a CRF receptor-1 agonist, or a pharmaceutically acceptable salt, complex or prodrug thereof, for the manufacture of a medicament for preventing or inhibiting neuronal cell death, in a mammal suffering from or susceptible to cerebral ischaemia, by stimulating CRF receptor-1.

8. The use as claimed in claim 5, 6 or 7, wherein the CRF receptor-1 agonist is a selective CRF receptor-1 agonist which binds to the CRF receptor-1 at least five times as strongly as it does CRF receptors-2α and/or -2β.

9. The use as claimed in claim 8, wherein the CRF receptor-1 agonist is a selective CRF receptor-1 agonist which binds to and stimulates the CRF receptor-1 at least five times as strongly as it does CRF receptors-2α and/or -2β.

10. The use of a CRF receptor agonist, or a pharmaceutically acceptable salt, complex or prodrug thereof, for the manufacture of a medicament for the prevention or inhibition of apoptotic neuronal cell death.

11. The use of a CRF receptor agonist, or a pharmaceutically acceptable salt, complex or prodrug thereof, for the manufacture of a medicament for the prevention or inhibition of neuronal cell death potentiated by inhibition or suppression of the PI 3-kinase signalling pathway.

12. The use as claimed in claim 10 or 11 wherein the neuronal cell death is potentiated by reduced expression, reduced activation, inhibition and/or deactivation of PI 3-kinase present in the neuronal cells.

13. The use of a CRF receptor agonist, or a pharmaceutically acceptable salt, complex or prodrug thereof, for the manufacture of a medicament for preventing or inhibiting neuronal cell death by stimulating or activating the PI 3-kinase signalling pathway.

14. The use of a CRF receptor agonist, or a pharmaceutically acceptable salt, complex or prodrug thereof, for the manufacture of a medicament for preventing or inhibiting neuronal cell death at least in part by suppression of GSK-3 present in the neuronal cells.

15. The use as claimed in claim 14 wherein the GSK-3 is suppressed by inhibition, in particular by phosphorylation.

16. The use as claimed in any one of claims 10 to 15 wherein the medicament is for the prevention or inhibition of neuronal cell death in a mammal suffering from or susceptible to chronic neurodegenerative disease, traumatic (mechanical) neuronal injury, epilepsy-associated neuronal loss, paralysis or spinal chord injury.

17. The use as claimed in claim 16 wherein the mammal is human and is suffering from or susceptible to Alzheimer's disease, Parkinson's disease or Huntington's disease.

18. The use as claimed in any one of claims 10 to 17, wherein the CRF receptor agonist is a CRF receptor-1 agonist, and the neuronal cell death is prevented or inhibited by stimulating CRF receptor-1.

19. The use as claimed in claim 18, wherein the medicament is for preventing or inhibiting neuronal cell death, in a mammal suffering from or susceptible to cerebral ischaemia, by stimulating CRF receptor-1.

20. The use as claimed in claim 18 or 19, wherein the CRF receptor-1 agonist is a selective CRF receptor-1 agonist which binds to the CRF receptor-1 at least five times as strongly as it does CRF receptors-2α and/or -2β.

21. The use as claimed in any one of claims 1 to 20 wherein the prevention or inhibition of neuronal cell death is potentiated by increasing the levels of intracellular cAMP in the neuronal cells.

22. The use as claimed in any one of claims 1 to 21, wherein the CRF receptor agonist or CRF receptor-1 agonist comprises CRF, urocortin, sauvagine or urotensin 1, or a pharmaceutically acceptable salt, complex or prodrug thereof.

23. The use as claimed in any one of the preceding claims, wherein the medicament is for administering to a/the mammal at a time of 30 mins to 8 hours, preferably 30 mins to 4 hours, after an acute neurodegenerative or potentially neurodegenerative occurrence.

24. A method of preventing or inhibiting neuronal cell death in a mammal suffering from or susceptible to chronic neurodegenerative disease, traumatic (mechanical) neuronal injury, epilepsy-associated neuronal loss, paralysis, or spinal chord injury, comprising administering to the mammal an effective amount of a CRF receptor agonist or a pharmaceutically acceptable salt, complex or prodrug thereof.

25. A method as claimed in claim 24 wherein the mammal is human and is suffering from or susceptible to Alzheimer's disease, Parkinson's disease or Huntington's disease.

26. A method of repairing or regenerating neuronal cells in a mammal in need thereof, comprising administering to the mammal an effective amount of a CRF receptor agonist or a pharmaceutically acceptable salt, complex or prodrug thereof.

27. A method of preventing or inhibiting apoptotic neuronal cell death in a mammal, comprising administering to the mammal an effective amount of a CRF receptor agonist, or a pharmaceutically acceptable salt, complex or prodrug thereof.

28. A method of preventing or inhibiting neuronal cell death in a mammal, the cell death being potentiated by inhibition or suppression of the PI 3-kinase signalling pathway, comprising administering to the mammal an effective amount of a CRF receptor agonist, or a pharmaceutically acceptable salt, complex or prodrug thereof.

29. A method of preventing or inhibiting neuronal cell death in a mammal by stimulating or activating the PI 3-kinase signalling pathway, comprising administering to the mammal an effective amount of a CRF receptor agonist, or a pharmaceutically acceptable salt, complex or prodrug thereof.

30. A method of preventing or inhibiting neuronal cell death in a mammal at least in part by suppression of GSK-3 present in the neuronal cells, comprising administering to the mammal an effective amount of a CRF receptor agonist, or a pharmaceutically acceptable salt, complex or prodrug thereof.

31. A method as claimed in any one of claims 24, 26, 27, 28, 29 or 30, wherein the CRF receptor agonist is a CRF receptor-1 agonist, and the neuronal cell death is prevented or inhibited, or the neuronal cells are repaired or regenerated, by stimulating CRF receptor-1.

32. A method of preventing or inhibiting neuronal cell death in a mammal suffering from or suceptible to cerebral ischaemia, comprising stimulating type-1 CRF receptors (CRF receptor-1) in the mammal by administering to the mammal an effective amount of a CRF receptor-1 agonist, or a pharmaceutically acceptable salt, complex or prodrug thereof.

33. A method as claimed in claim 31 or 32, wherein the CRF receptor-1 agonist is a selective CRF receptor-1 agonist which binds to the CRF receptor-1 at least five times as strongly as it does CRF receptors-2α and/or -2β.
